(19) United States
(12) Patent Application Publication
(10) Pub. No.: US 2025/0034614 A1
(43) Pub. Date: Jan. 30, 2025

(54) PERSISTER CONTROL BY LEVERAGING DORMANCY ASSOCIATED REDUCTION OF ANTIBIOTIC EFFLUX

(71) Applicants: Dacheng REN, Manlius, NY (US); Sweta ROY, Woodside, NY (US)

(72) Inventors: Dacheng REN, Manlius, NY (US); Sweta ROY, Woodside, NY (US)

(73) Assignee: Syracuse University, Syracuse, NY (US)

(21) Appl. No.: 18/710,390

(22) PCT Filed: Nov. 15, 2022
(86) PCT No.: PCT/US22/49909

§ 371 Date: May 15, 2024

Related U.S. Application Data
(60) Provisional application No. 63/279,290, filed on Nov. 15, 2021.

(51) Int. Cl. C12Q 1/18; A61K 31/343; A61K 31/65; A61K 45/06; A61P 31/04

(52) U.S. Cl. CPC C12Q 1/18; A61K 31/343; A61K 31/65; A61K 45/06; A61P 31/04

(57) ABSTRACT

A strategy for bacterial persister control using amphiphilic antibiotics that do not require active transport to penetrate bacterial membranes of persister cells. Persister cells have reduced drug efflux and accumulate more minocycline than normal cells, leading to effective killing of this dormant subpopulation upon wake-up. While dormancy is a well-known cause of antibiotic tolerance, it also provides an Achilles' heel for controlling persister cells by leveraging dormancy associated reduction of drug efflux.

### CROSS-REFERENCE TO RELATED APPLICATIONS

This present application claims priority to U.S. Provisional Application No. 63/279,290, filed on Nov. 15, 2021.

### STATEMENT REGARDING FEDERALLY SPONSORED RESEARCH OR DEVELOPMENT

This invention was made with government support under Grant No. 1706061 awarded by the National Science Foundation (NSF). The government has certain rights in the invention.

### BACKGROUND OF THE INVENTION

### 1. Field of the Invention

The present invention relates to the treatment of bacterial infections and, more particularly, to an approach for reducing antibiotic resistance attributable to dormant cells, especially persister cells.

### 2. Description of the Related Art

Despite the past decades of success in infection control by antibiotics, persistent bacterial infections remain challenging in tuberculosis, Lyme disease, and those associated with cystic fibrosis and implanted medical devices. These seemingly different disease conditions share the same root cause, bacterial dormancy. It is well documented that bacteria form persister cells, a small subpopulation of dormant phenotypic variants that are highly tolerant to different stresses including antibiotics. Persister cells are growth-arrested, but can restart growth when the external stress is removed, causing relapse of infection. In addition, treatment of persistent infections causes overuse of antibiotics, contributing to the development of antibiotic resistance. Therefore, a strategy to eradicate these dormant populations is urgently needed.

The molecular mechanism of persister formation has been under intensive debate and currently there is no consensus model. But it is well known that persister cells can form both stochastically and via triggering by stressors such as antibiotic treatment, starvation, limited carbon source, host factors, and oxidative stress. Unlike antibiotic resistant strains that have genetic elements to degrade or extrude antibiotic molecules, or alter the drug target, persister cells do not degrade antibiotics and still have the drug target. However, due to inactive metabolism, the binding of an antibiotic to its target does not generate corrupted products that lead to cell death as occurs in normal cells. This has been overlooked in antibiotic drug discovery over the past decades based on the Waksman platform. This platform selects leads for growth inhibition; thus, it is not surprising that most available antibiotics are ineffective against dormant persister cells. In addition to the challenge in persister killing by antibiotic molecules, persister formation also presents barriers to membrane penetration by many antibiotics. Dormancy is accompanied by significant reduction in membrane potential and proton motive force, which blocks the penetration of antimicrobials that rely on active uptake. Even for antibiotics that enter cells by energy-independent diffusion through porins, the decrease in membrane potential reduces the ion motive force for positively charged molecules, making it less favorable for drug influx. Thus, this subpopulation survives antibiotic treatment and gives rise to its progeny population that is genetically identical to the original population after the antibiotic stress is removed, leading to persistent infections.

Persister cells are metabolically inactive, and thus lack growth-associated targets of most antibiotics. One possible strategy to overcome this challenge is to find agents that can kill the persister population directly. Mitomycin C and cisplatin have been shown to crosslink the DNA and kill persister cells. Specifically, mitomycin C can enter cell passively and crosslinks guanine bases on different DNA strands, while cisplatin crosslinks the purines. In addition, cisplatin contains a platinum ion, which may contribute to the production of ROS. Mitomycin C showed promising activities for topical use in an in vitro wound infection model. Meanwhile, there are reports of toxicity of cisplatin and mitomycin C at high concentrations when administered intravenously for cancer treatment.

With direct killing of persister cells being difficult, another strategy that has been explored is to address the challenge of dormancy associated reduction of antibiotic penetration. Gram-negative bacteria are particularly challenging due to the presence of an outer membrane (OM) composed of anionic lipid polysaccharides. In general, hydrophilic antibiotics can gain access to the cell interior through porins in the OM, while hydrophobic molecules can enter through the lipid bilayer. Dormancy is accompanied by significant reduction in membrane potential, which blocks the penetration of antimicrobials that rely on active uptake. Even for antibiotics that enter cells by energy-independent diffusion through porins, the decrease in membrane potential reduces the ion motive force for positively charged molecules, making it less favorable for drug influx.

A few strategies have been reported to promote penetration of antibiotics, primarily aminoglycosides, into persister cells. These strategies include increasing aminoglycoside uptake through hypoionic shock, generating proton motive force (PMF) with metabolites and conjugating tobramycin with a membrane targeting peptide. For example, researchers have demonstrated that it is possible to kill persister cells with internalized gentamycin during wake-up with resumed central metabolism, but not full growth activities. However, these strategies require potentiation with sugar or hypoionic shock, which can be difficult to apply in vivo.

### BRIEF SUMMARY OF THE INVENTION

The present invention is a new strategy of persister control and demonstrates that minocycline, an amphiphilic antibiotic that does not require active transport to penetrate bacterial membranes, is effective in killing Escherichia coli persister cells [by 70.8±5.9% (0.53 log) at 100 μg/mL], while being ineffective in killing normal cells. As a result, the present invention can be used to identify effective control agents that do not require pretreatment and provide a mechanism for using those control agents to treat infections. The present invention was initially demonstrated with minocycline and rifamycin. These two antibiotics are substrates of E. coli efflux pumps and thus ineffective against the normal cells of E. coli. However, because efflux requires proton motive forces (PMF), efflux should be inactive in persisters. As a result, favorable conditions existed for antibiotic accumulation and persister killing during wake-up. Testing demonstrated that minocycline treatment according to the present invention could be effective in killing E. coli persister cells. This led to a set of principles for identifying persister control agents, which were validated by testing eravacycline, which has a stronger binding to its target than minocycline, and which demonstrated that eravacycline is more potent in killing E. coli persister cells than minocycline (3 logs vs. 0.5 log of killing at 100 μg/mL).

Further mechanistic studies revealed that persister cells have reduced drug efflux and accumulate more minocycline than normal cells, leading to effective killing of this dormant subpopulation upon wake-up. Consistently, eravacycline, which also targets the ribosome but has a stronger binding affinity than minocycline, kills persister cells by 3 logs when treated at 100 μg/mL. Additional results demonstrate that this new strategy is also effective against persister cells of Pseudomonas aeruginosa and its biofilms. Based on the results, a set of new criteria for selecting persister drugs is developed. Screening of a small compound library using k-means clustering identified additional agents for persister control. In summary, the present invention reveals that while dormancy is a well-known cause of antibiotic tolerance, it also provides an Achilles' heel for controlling persister cells by leveraging a dormancy-associated reduction of drug efflux to load persister cells and then cause killing by waking up the persisters.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a schematic of persister control by leveraging reduced antibiotic efflux. Persisters have reduced membrane potential and thus are difficult to penetrate by hydrophilic antibiotics and those require active transport. In comparison, antibiotics that can penetrate through lipid without active uptake can still target persister cells. Additionally, reduced drug efflux provides a favorable condition for accumulation of antibiotics in persister cells. This leads to killing if the internalized antibiotic molecules remain bound to the target during wake-up. The inactivated pathways in persister cells are indicated with lighter colors and/or marked with “X”. Minocycline, rifamycin SV, and eravacycline fit the criteria and are found effective in this study for persister control.

FIG. 2 is a graph of the viability of E. coli HM22 persister and normal cells after tetracycline treatment.

FIG. 3 is a graph of the effects of minocycline on the viability of normal (black bars) and persister (white bars) cells of E. coli HM22. The untreated samples from each population were normalized as 100%. Means±SE are shown (n=5).

FIG. 4 is a graph of the different antibiotic treatments of E. coli HM22 persister cells including 100 μg/mL of ampicillin, 100 μg/mL of minocycline, and the combination of both.

FIG. 5 is a table of the intracellular concentration of minocycline based on the reporter bioassay where minocycline concentration was calculated using a standard curve of reporter strain for each population and means±SE are shown (n=4).

FIG. 6 is a graph of the intracellular concentration of minocycline from treated and untreated samples in both normal (black bars) and persister (patterned bars) populations using LC-MS. Means±SE are shown (n=3). *p-value≤0.05, **p-value 0.01, ***p-value 0.001, ****p-value 0.0001.

FIG. 7 is a schematic overview of the lysate collection after both normal and persister cells of E. coli HM22 were treated with minocycline.

FIG. 8 is a graph of showing that the reporter strain, B. subtilis 168, was used to evaluate the killing activities of cell lysates.

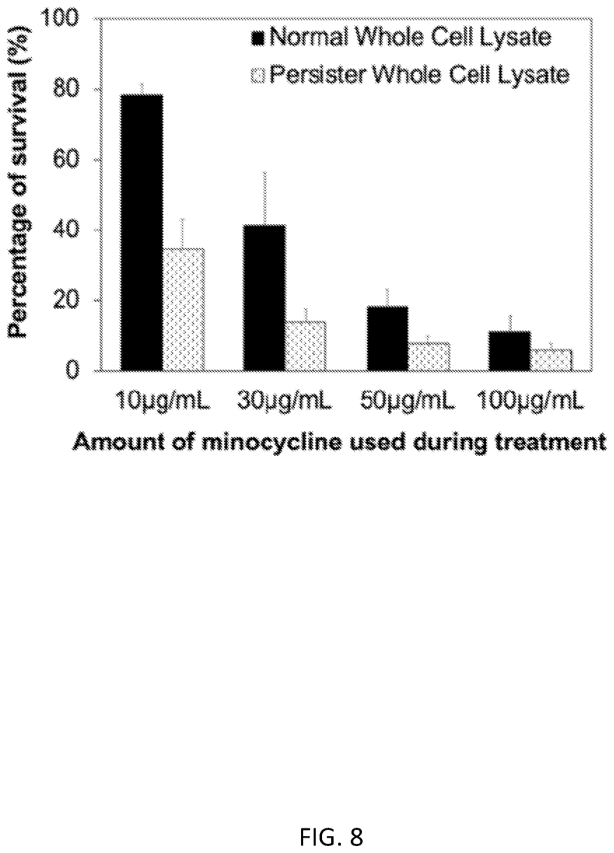

FIG. 9 is a graph of a standard curve for quantification of antibiotic concentration in unknown samples.

FIG. 10 is a schematic showing that the concentration obtained from the standard curve and killing activity was normalized by the number of cell and cell volume.

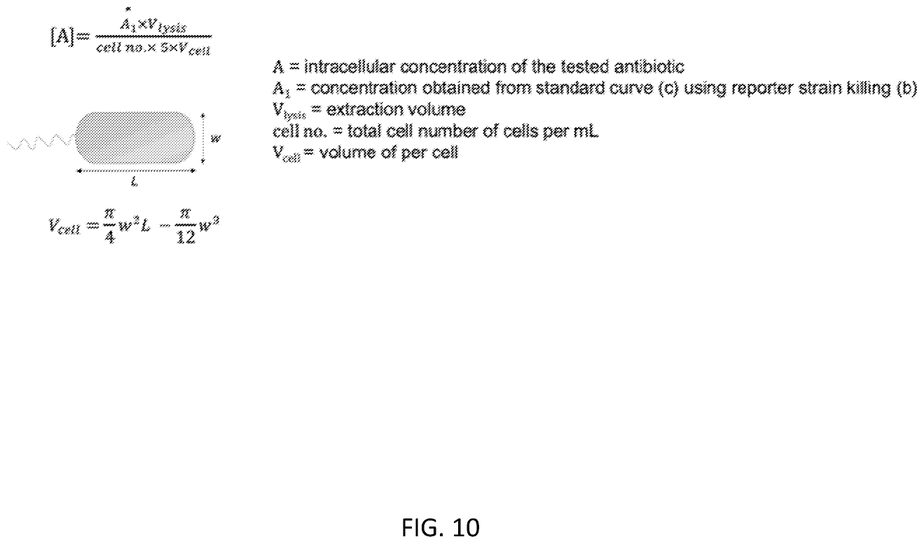

FIG. 11 is a graph of the induction of persister formation led to increased EtBr accumulation. Measurements were performed with a fluorescence microplate reader (Model FLx800, Bio-Tek Instruments, Winooski, VT, USA) with excitation at 360 nm.

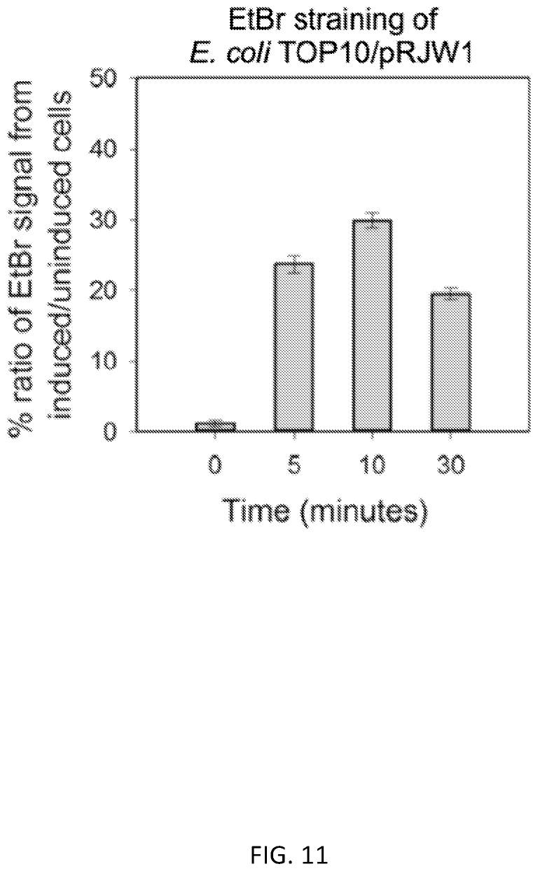

FIG. 12 is a series of graphs of the flow cytometry analysis of EtBr staining showing EtBr stained uninduced E. coli Top10/pRJW1 (top) and EtBr stained arabinose induced E. coli Top10/pRJW1 (bottom); EtBr stained uninduced E. coli Top10 pBAD (top) and EtBr stained arabinose induced E. coli Top10/pBAD (empty vector) (bottom); E. coli ΔacrB without (top) and with (bottom) EtBr staining; E. coli pUC19-acrB without (top) and with (bottom) EtBr staining (from left to right).

FIG. 13 is a graph of the inactivation of efflux pumps sensitized normal cells to minocycline. Means±SE are shown (n=3). *p-value<0.05, **p-value <0.01, ***p-value <0.001, ****p-value 0.0001.

FIG. 14 is a series of schematics and accompanying graphs of hipA mediated persister formation with induction of arabinose and tetracycline. Flow cytometry analysis of JC-1 stained samples was used to compare the membrane potential of induced E. coli Top10/pRJW1 (top) vs. induced E. coli Top10/pBAD (empty vector) (bottom) cells. A shift to low red fluorescence was observed for 16% of induced cells of E. coli Top10/pRJW1, while no change was observed in green fluorescence.

FIG. 15 is a graph of persister count increased when induced with both arabinose and tetracycline.

FIG. 16 is a graph of E. coli HM22 normal population was pretreated with CCCP (100 μM) to reduce the membrane potential. The cells were then treated with 100 pg/mL of minocycline. Means±SE are shown (n=3).

FIG. 17 are representative fluorescent images of control and minocycline treated E. coli HM22 normal cells with or without CCCP pretreatment. The cells were labeled with SYTO9 and propodium iodide (PI) (scale bar, 10 μM);

FIG. 18 is a pair of graphs of mean fluorescence intensity of SYTO9 and PI quantified using Image J. *p-value<0.05, **p-value 0.01, ***p-value 0.001, ****p-value 0.0001.

FIG. 19 is a series of representative fluorescence images of control and minocycline treated E. coli HM22 normal and persister cells after LIVE/DEAD staining (scale bar=10 μm).

FIG. 20 is a pair of graphs of showing that fluorescence signals were used to compare the viability of normal (left) and persister (right) cells. Mean fluorescence intensity of SYTO9 and PI was quantified using ImageJ.

FIG. 21 is a series of representative fluorescence images of persister cells upon wake-up after minocycline treatment. The images show persister and normal cells at 0 and 30 min after spiking with Lysogeny broth (LB) medium.

FIG. 22 is a graph of fluorescence signals of LB spiked E. coli HM22 persister cells. Three biological replicates were tested with 16 images randomly analyzed from each sample;

FIG. 23 is a graph of OD₆₀₀ of E. coli HM22 persister cells during wake-up. Cells with and without minocycline treatment were compared.

FIG. 24 is a graph of the intracellular concentration of minocycline after minocycline was removed from the solution and replaced with LB medium. *p-value<0.05, **p-value 0.01, ***p-value 0.001, ****p-value 0.0001.

FIG. 25 is a graph of the effects of 100 μg/mL rifamycin SV on the viability of normal (black bars) and persister (patterned bars) cells of E. coli HM22. Means±SE are shown (n=3).

FIG. 26 is a graph of the intracellular concentration of rifamycin SV based on the reporter strain bioassay. Rifamycin SV concentration was calculated using the standard curve of reporter strain for each population. Means±SE are shown (n=3).

FIG. 27 is a graph of the effects of eravacycline on the viability of normal (black bars) and persister (patterned bars) cells of E. coli HM22. Means±SE are shown (n=3).

FIG. 28 is a graph of the intracellular concentration of eravacycline based on the reporter strain bioassay. Eravacycline concentration was calculated using a standard curve of reporter strain for each population. Means±SE are shown (n=3).

FIG. 29 is a graph of the different antibiotic treatments of E. coli HM22 persister cells including 100 μg/mL of ampicillin, 100 μg/mL of eravacycline, and the combination of both. Means±SE are shown (n=3).

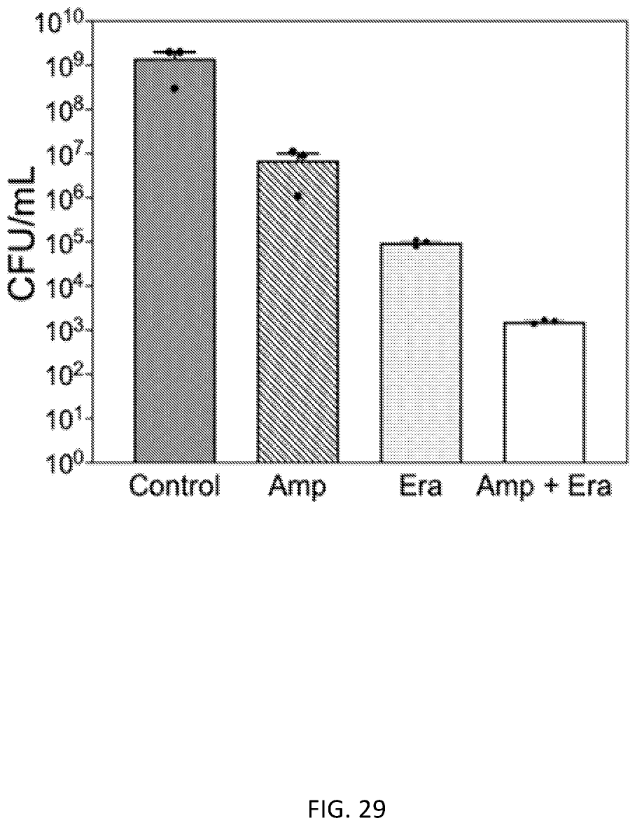

FIG. 30 is a graph of the relative number of viable E. coli HM22 persister cells after eravacycline treatment (initial number normalized as 100%). The changes in OD₆₀₀ (g) and CFU (h) were followed over time. Means±SE are shown (n=3 for OD₆₀₀ and n=4 for CFU). *p-value<0.05, **p-value <0.01, ***p-value <0.001, ****p-value 0.0001.

FIG. 31 is a graph of the relative number of viable E. coli HM22 persister cells after eravacycline treatment (initial number normalized as 100%).

FIG. 32 is a graph of the standard curve of tetracycline, where the standard curve of tetracycline was generated using the reporter strain E. coli ΔtolC treated with E. coli HM22 lysates supplemented with known concentrations of tetracycline.

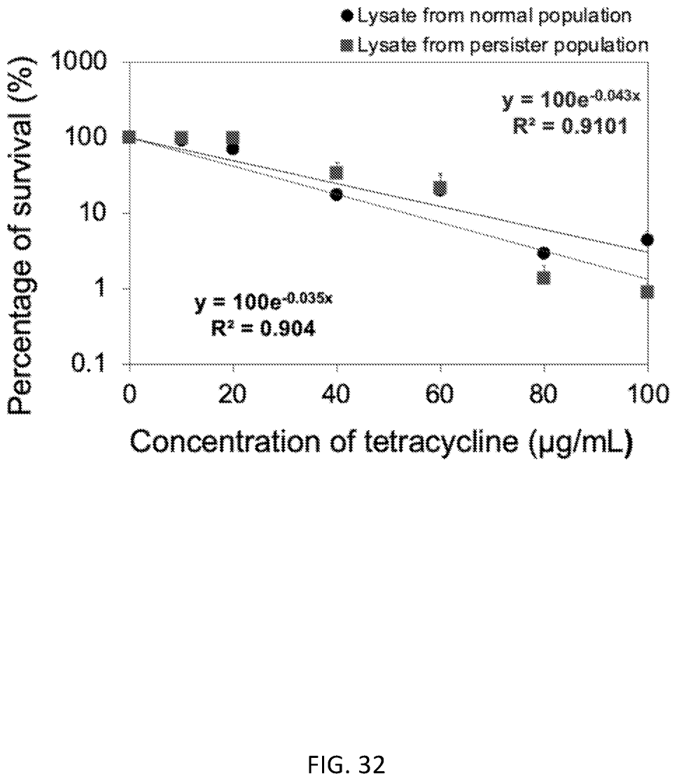

FIG. 33 is a graph of the standard curve of rifamycin SV generated using the reporter strain B. subtilis 168 treated with E. coli HM22 lysates supplemented with known concentrations of rifamycin SV.

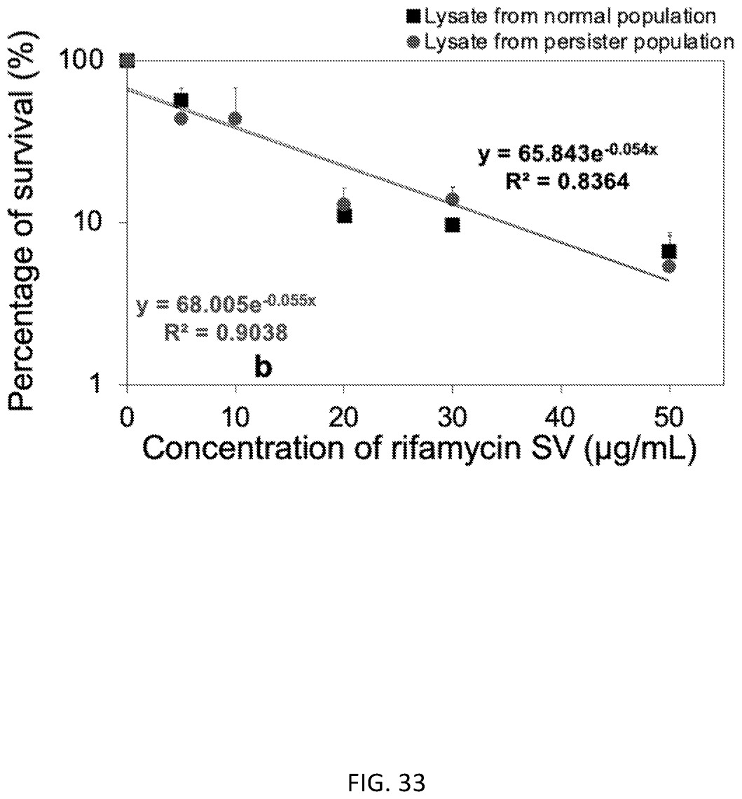

FIG. 34 is a graph of the standard curve of eravacycline, which was generated using the reporter strain S. aureus ALC2085 treated with E. coli HM22 lysates supplemented with known concentrations of eravacycline.

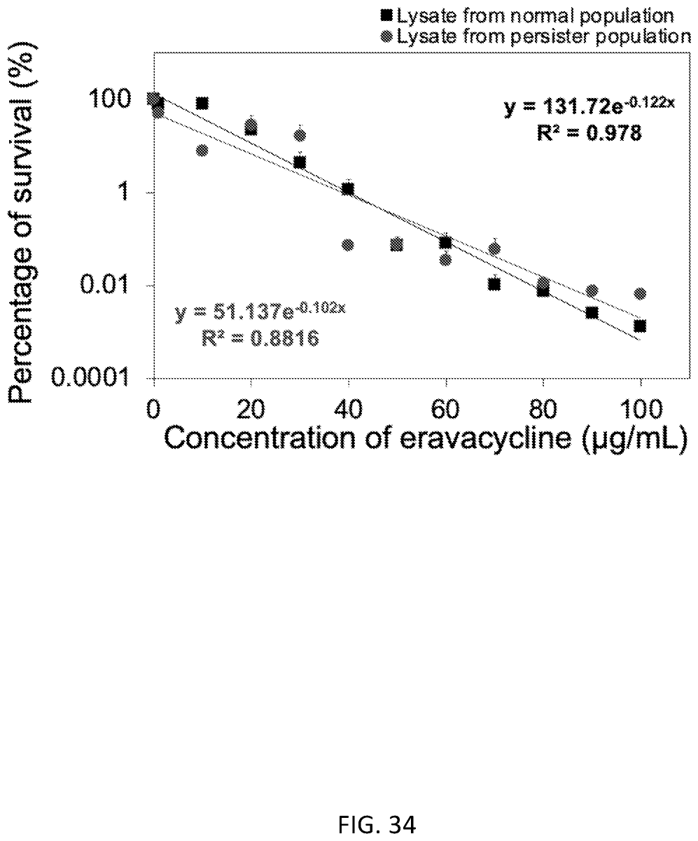

FIG. 35 is a graph of the inactivation of efflux pumps sensitized normal 18 cells to eravacycline. The graph shows the CFUs after eravacycline treatment of E. coli BW25113, E. coli BW25113 ΔacrA, E. coli BW25113 ΔtolC, and E. coli BW25113 ΔacrB. Means±SE are shown (n=3).

FIG. 36 is a series of graph showing the effects of eravacycline on Pseudomonas aeruginosa wildtype, PAO1 and its mucoid strain, PDO300. Viability of exponential phase (grey bars) and carbonyl cyanide m-chlorophenyl hydrazone (CCCP) isolated PAO1 persister cells (dark grey bars) after treatment with different concentrations of eravacycline. Persister cells were isolated after 3 h treatment with CCCP and washed to remove the antibiotic. Treatment was done in phosphate buffered solution for 1 h. Means SE are shown (n≥3)

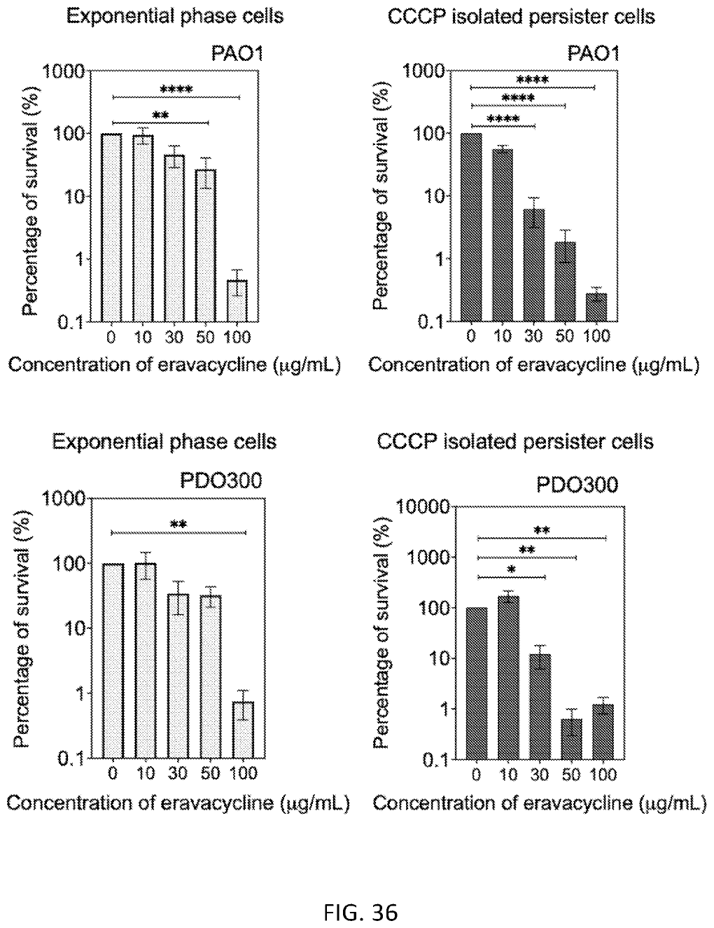

FIG. 37 shows 48 h (a-c) PAO1 and (d-f) PD0300 biofilms on PDMS surfaces were grown and treated with different concentrations of eravacycline for 1 h. Viability of biofilm cells was determined by counting CFU (a & d). 48 h PAO1 and PDO300 biofilm on PDMS surfaces were treated with 100 μg/mL of eravacycline for 1 h and imaged after labeling with LIVE/DEAD staining (b & e). (d) Biomass was quantified using COMSTAT (c & f). Means±SE are shown (n=3).

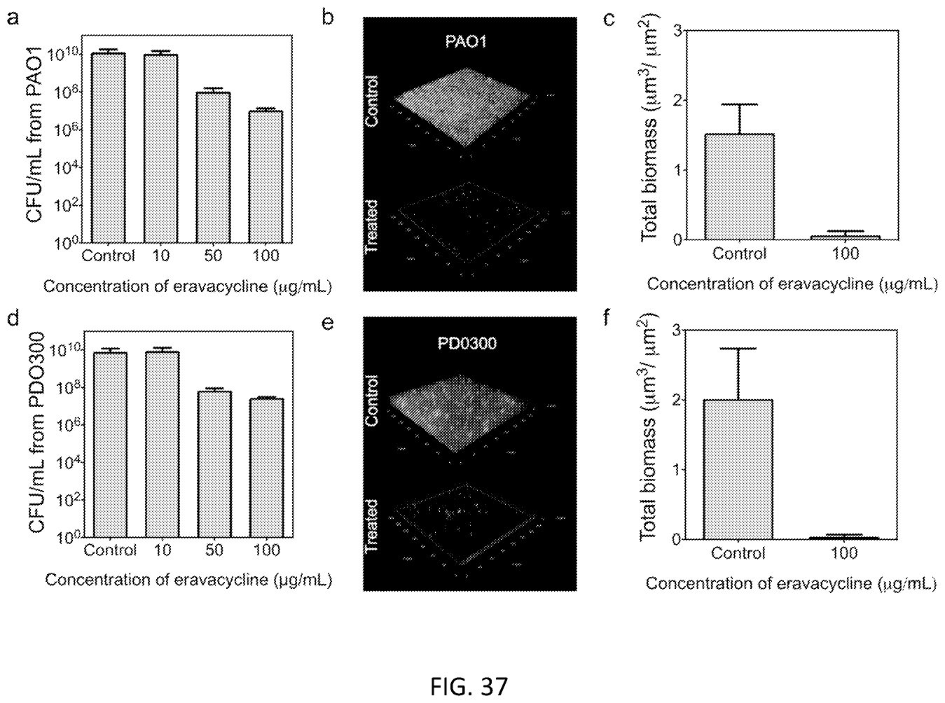

FIG. 38 is a pair of graphs of K-means clustering that led to identification of potential persister control agents. (a) Using log P, lipophilicity of the molecules similar to that of the positive candidates can be identified. By clustering this parameter, we have identified 14 candidates whose log P values are similar to that of minocycline and eravacycline. (b) Clustering the number of halogen atoms and aliphatic OH identified 13 molecules similar to that of eravacycline.

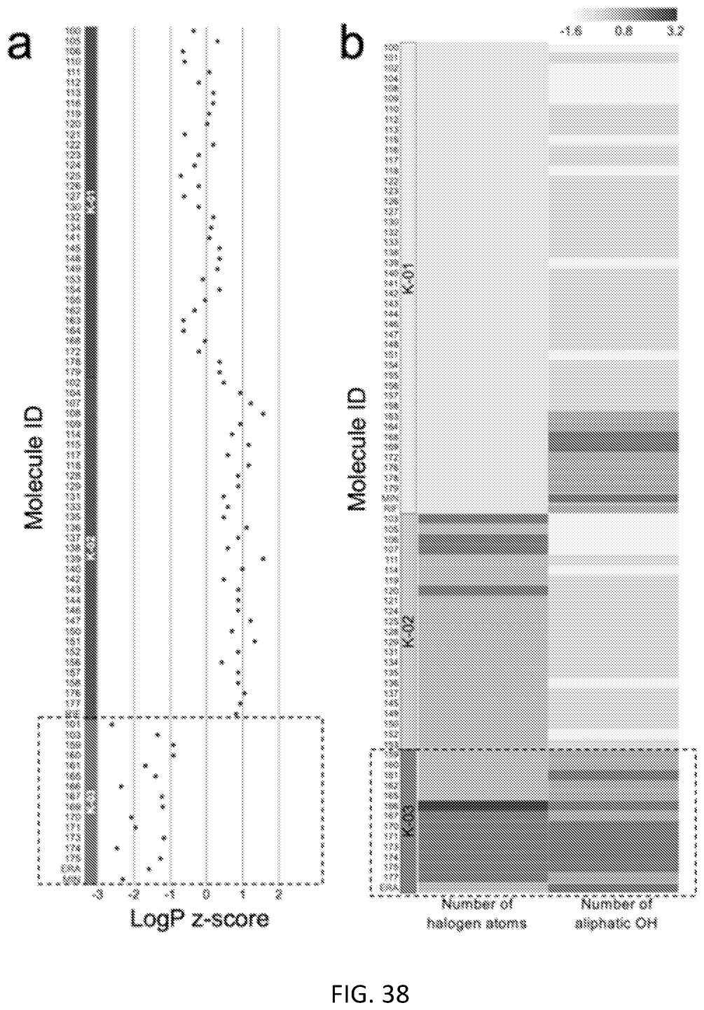

FIG. 39 is a series of derivatives of the positively screened compound 161, which may have activities against persister cells.

FIG. 40 is a series of graphs of the screening for potential persister control agents. The clustering results led to identification of 10 compounds with similar chemical properties to rifamycin and minocycline. Experimental testing revealed that compounds 161 and 173 are potent persister control agents killing 95.5±1.7% and 94.2±1.4% of the persister population, respectively.

FIG. 41 is a series of compounds that have been clustered with their IUPAC names.

FIG. 42 is a pair of graphs of showing that compounds 161 and 173 are new persister control agents. Graphs show the effects on normal (white bars) and persister cells (grey bars) using different concentration of compounds 161 (a) and 173 (b)

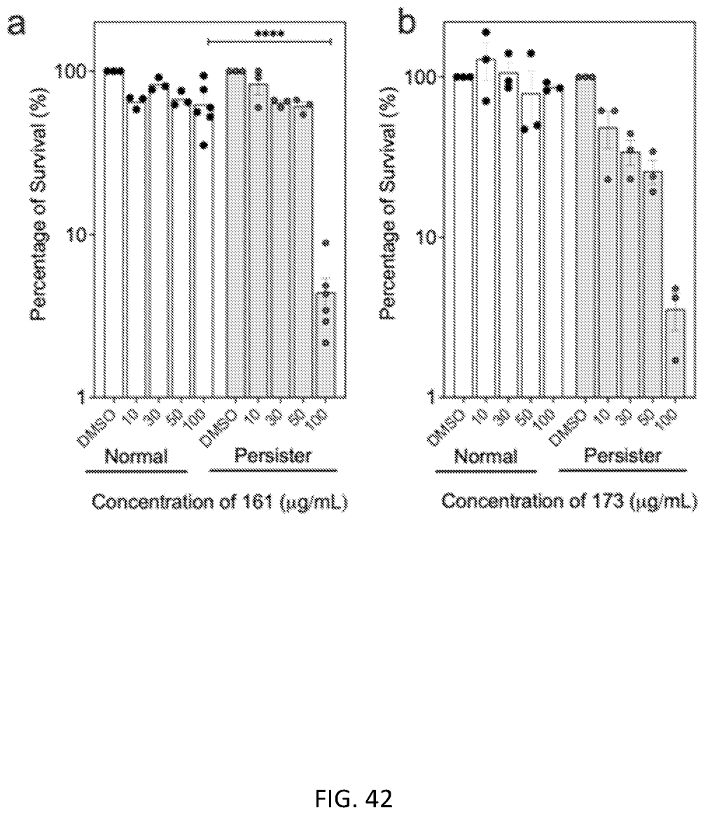

FIG. 43 is pair of graphs showing that compounds 161 and 173 are substrates of the RND efflux pump. Using a concentration of 50 μg/mL of compound 161 (a) and 173 (b), AcrAB-tolC efflux pump activity was evaluated.

FIG. 44 is a graph of the intracellular concentration of compound 161 based on the reporter strain bioassay (a) in both normal and persister population of E. coli HM22.

FIG. 45 is a series of derivatives of the positively screened compound 161, which may have activities against persister cells.

FIG. 46 is a graph that shows the viability of P. aeruginosa persisters cells (isolated using ciprofloxacin) treated either with 100 μg/mL of eravacycline (ERA), 100 g/mL of compound 123 or cotreatment of compound 123 and eravacycline with a concentration of 100 μg/mL of both. The cotreatment showed potent activity on P. aeruginosa persisters cells compared to the individual treatment.

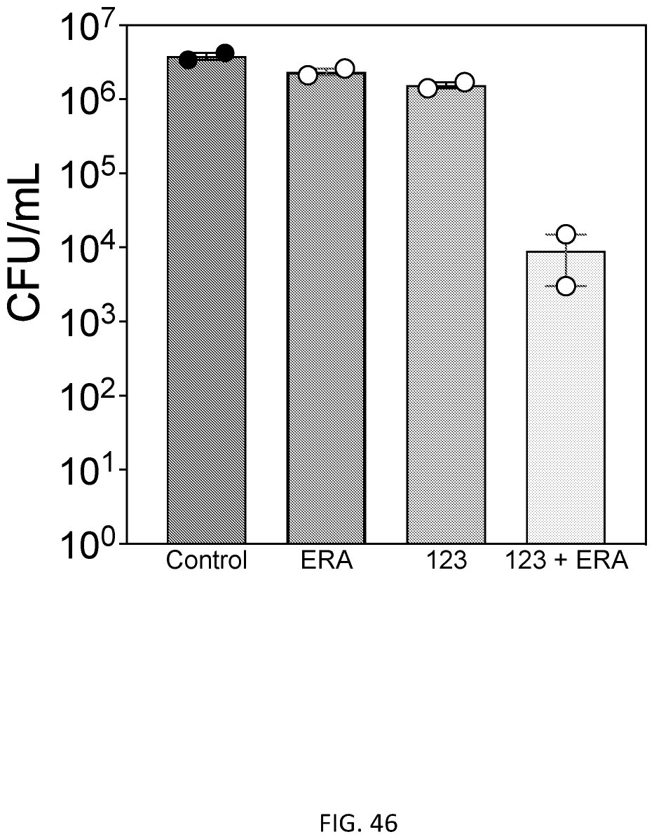

FIG. 47 is a table showing a series of derivatives of compound 123.

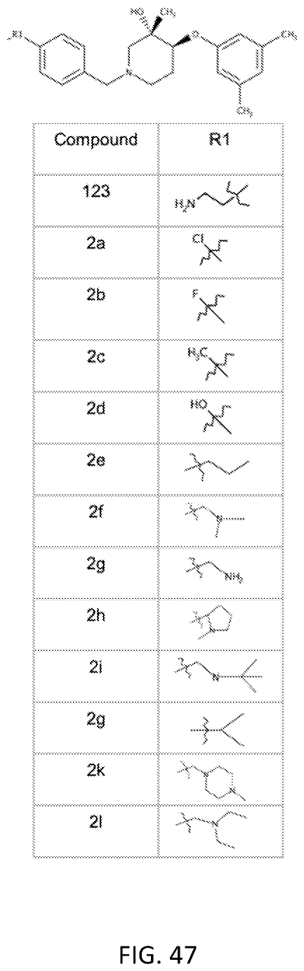

### DETAILED DESCRIPTION OF THE INVENTION

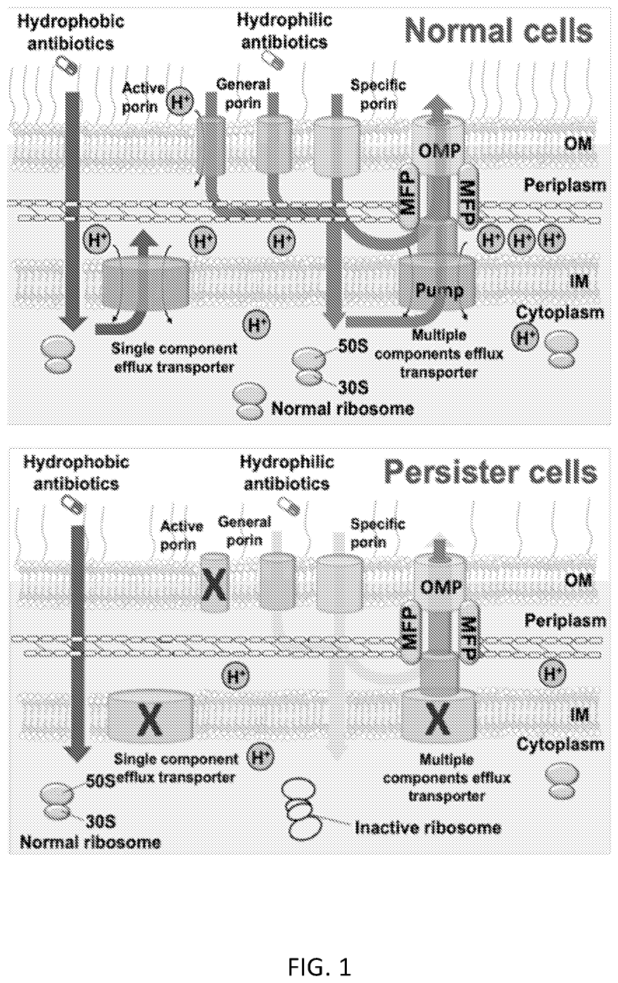

Referring to the figures, wherein like numerals refer to like parts throughout, there is seen in FIG. 1 a new approach for controlling persister cells by leveraging reduced antibiotic efflux according to the present invention. The present invention involves the effective treatment of bacterial persister cells that are otherwise tolerant to conventional antibiotics by selecting the right antibiotics with appropriate treatment conditions. Specifically, the present invention is based on the discovery that antibiotics capable of penetrating bacterial cells by energy-independent diffusion and binding to their target strongly can kill persister cells during wake-up. Thus, the decrease in membrane potential of persister cells provides an “Achilles' heel” for killing this dormant population. For example, the binding of minocycline with the ribosome is an energy-independent process regardless of the cell's physiological state. This is expected to occur in persister cells due to the ability of minocycline to diffuse through the membrane and avoid extrusion due to reduced efflux activities in persister cells that ultimately led to killing of this population.

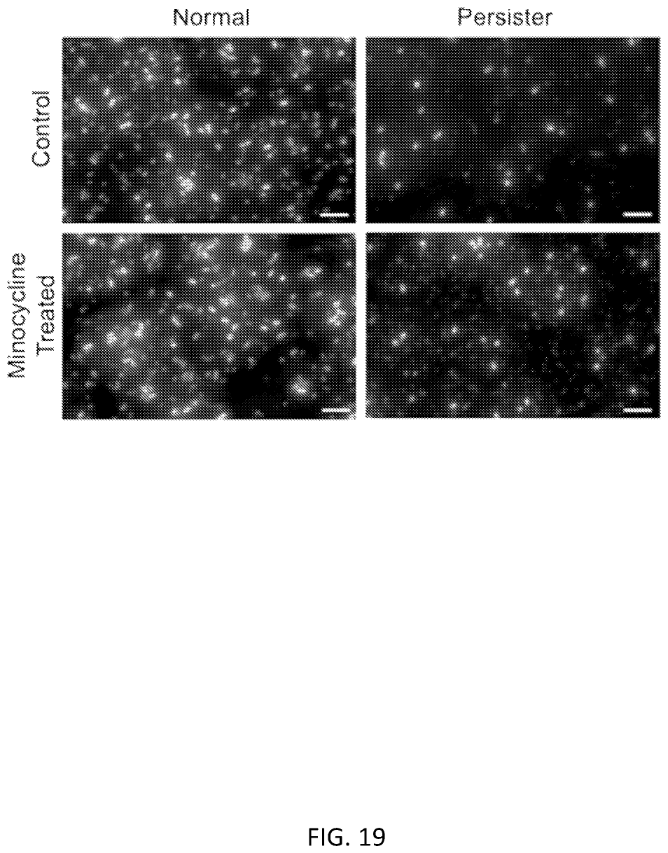

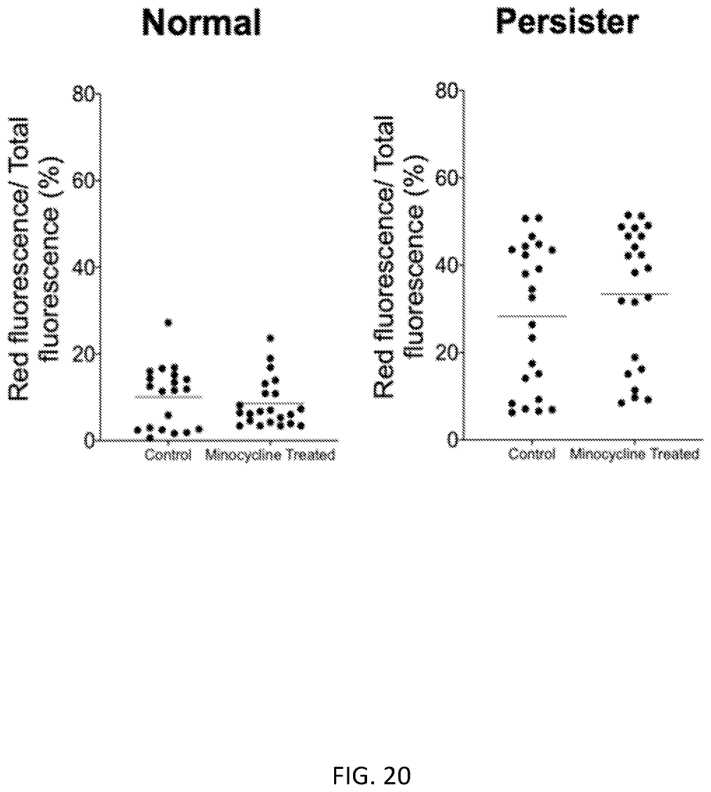

The present invention is accompanied by evidence that the killing did not occur instantly but wake-up is required for the activity to take place (FIG. 19 through 24). With the extracellular antibiotics removed and nutrients provided, persister cells can revert to normal cells, a process that requires transcriptional and translational activities. Minocycline and eravacycline both target the ribosome while rifamycin SV targets the RNA polymerase, leading to killing of persister cells during wake-up. This represents a new strategy for persister control.

A few criteria need to be satisfied for the strategy of persister control of the present invention to work. These include: (1) the control agent needs to penetrate persister cells via energy-independent diffusion (amphiphilic compounds preferred); (2) the target should be present in the persister cells; and (3) the control agent should have sufficient binding affinity with the target. Sufficient binding affinity refers to an amount of binding affinity to the target such that the compound will not be extruded or diffuse out of the cell. Minocycline, rifamycin SV and eravacycline all meet these criteria. It is worth noticing that normal cells of E. coli are resistant to these antibiotics due to substrate specific efflux activities energized by membrane potential gradient. In general, this is not favorable for bacterial control. However, these antibiotics provide a promising solution to the challenges of persister cells. This strategy is different from pulsed dosing of antibiotics that has recently been shown to improve the killing of biofilms and persister cells. It includes antibiotic-free periods between doses so that dormant cells can be killed after they resuscitate. However, pulsed dosing requires resuscitation but before overgrowth (to prevent the formation of new persisters), thus a narrow window between doses. In comparison, the method of the present invention is a different strategy that targets persister cells specifically. It does not need repeated dosing and kills persister cells before they fully resuscitate.

The results from this study of the present invention also emphasize the needs for new antibiotic discovery platforms. The vast majority of currently available antibiotics were discovered between 1940s-1960s using the Waksman platform. In this approach, a possible source of antimicrobials (e.g., a soil sample containing Actinomycetes) is tested for its inhibiting zone on an overlay plate against a target bacterial species. This approach selects lead compounds based on growth inhibition and thus the hits commonly fail to achieve persister control. Based on the findings from this study, future screenings based on membrane penetration may generate new leads that can better control dormant bacterial cells. If the compound is a substrate of efflux (only effective against persister cells), it may be applied with other antibiotics together to synergistically target both normal and dormant populations, e.g., synergy between ampicillin and eravacycline/minocycline found in this study.

Another important area for future development is target binding which includes both target selection and binding affinity. Although persister cells do not have genetic mutations, tolerance may induce phenotypic changes that reduce the availability of drug target. For example, the formation of persister cells is accompanied by suppression of protein production and thus a lower amount of key ribosomal proteins compared to normal cells. Furthermore, persister cells also contain inactive ribosomes (inactive 70S, 90S, and 100S ribosomes) as a means of preservation during stress. The findings herein showed that E. coli persister cells accumulated 2.6 times of minocycline compared to normal cells. However, since persister cells only contain about 25% of normal ribosomes compared to the normal cells, the ratio of intracellular minocycline molecules to the amount of target is probably more than 10 times higher. This helps explain the killing of persister cells since activation of ribosome complexes is a crucial step for persister resuscitation. Future studies are needed to identify drug candidates with strong target binding in both normal and persister cells to eradicate both populations.

Overall, the findings of the present invention demonstrate the feasibility to kill persister cells by antibiotics that can penetrate membranes through energy independent pathways (without active uptake) and have strong binding with the target. These agents can cause persister killing during “wake-up” when the extracellular stressors are removed. Developing more effective agents based on this strategy requires a better understanding of the structural effects of antimicrobial on persister killing. Identifying appropriate wake-up conditions is also important for further development of persister control strategies

### Example 1

Minocycline is effective in killing persister cells but not normal cells of E. coli.

It is generally believed that conventional antibiotics that can kill normal cells are ineffective against persister cells. To identify persister control agents, a different approach was used to test antibiotics that are ineffective against normal cells and are substrates of drug efflux pumps. Tetracycline and minocycline, both from the tetracycline family of antibiotics, were tested first. Both antibiotics target protein translation by binding to the ribosome complex and are substrates of the resistance-nodulation-cell division (RND) efflux pumps. The RND efflux pumps require proton motive force (PMF) to function and are involved in pumping out multiple agents such as antibiotics and toxins.

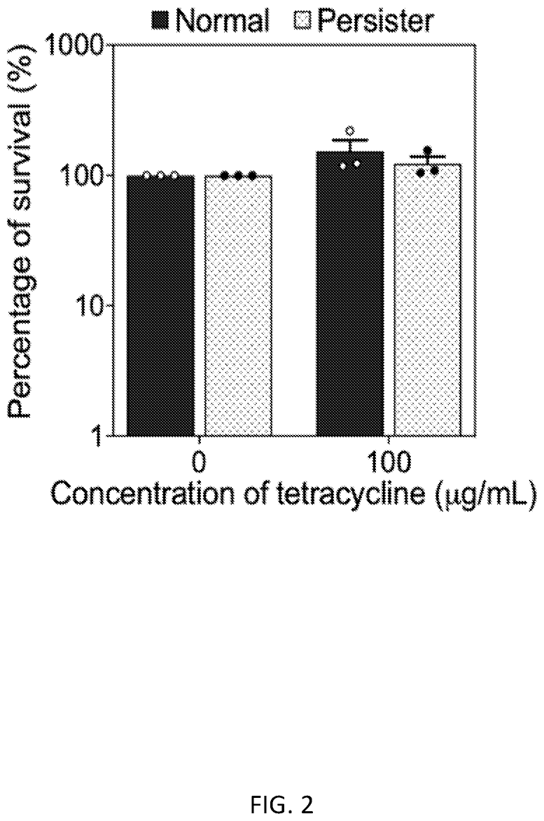

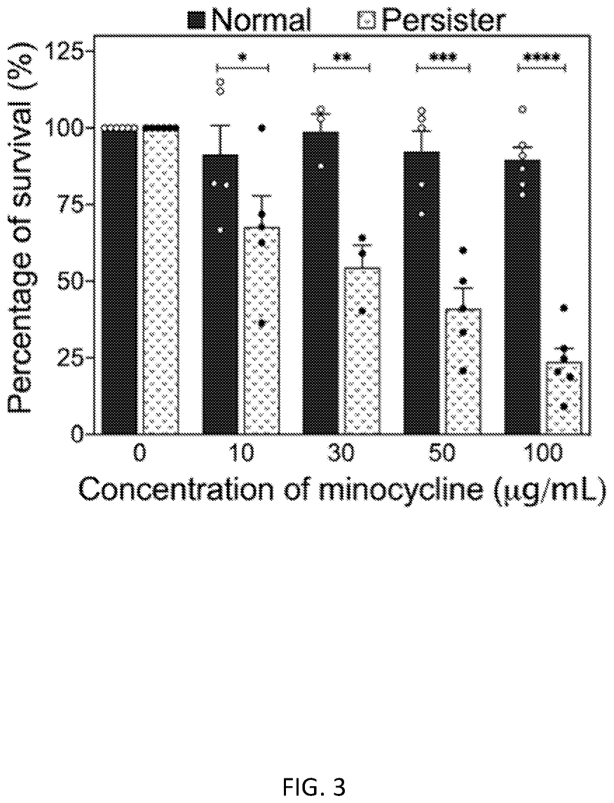

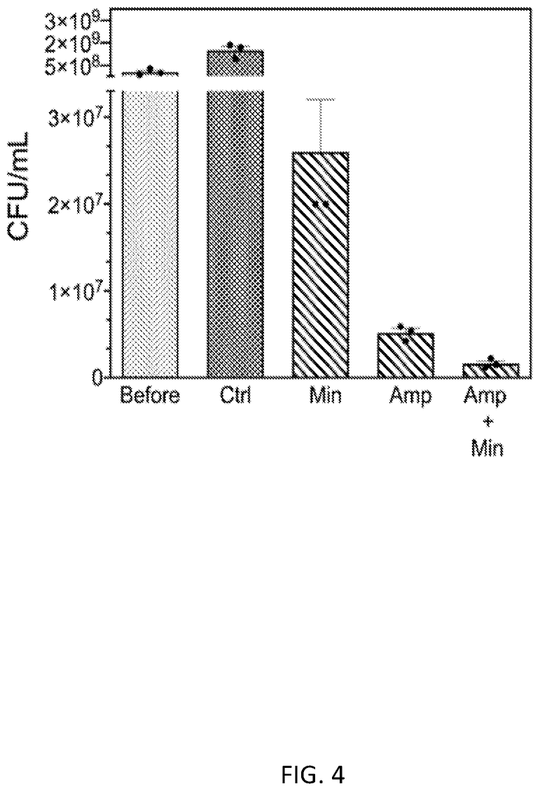

E. coli HM22 was used as the model strain in this test because it contains the hipA7 allele that leads to high-level persistence. First, E. coli HM22 cells were treated in exponential phase (˜99% as normal cells) and persister cells isolated with ampicillin. Both exponential phase cells and persister cells were tolerant to tetracycline (FIG. 2). However, they responded differently to minocycline treatment. As expected, even at a high concentration of 100 μg/mL, there was no significant killing (FIG. 2) of normal cells (cells from exponential cultures) by minocycline. In contrast, exposure to minocycline killed 32.3 9.1% (p=0.030), 47.8±5.3% (p=0.047), 59.0±6.0% (p<0.001), and 70.8±5.9% (p<0.001) of isolated persister cells when treated at 10, 30, 50, and 100 μg/mL, respectively (FIG. 3). This demonstrated persister killing by minocycline in a dose-dependent manner. Because the persister cells were isolated using ampicillin, ampicillin was evaluated to determine if it played a role in the increased killing of persister cells by minocycline. To test this, the treatment with 100 μg/mL ampicillin alone was compared with concurrent treatment with both 100 μg/mL ampicillin and 100 μg/mL minocycline in LB. The results showed that adding minocycline caused an additional 68% of killing (p=0.0038, unpaired t-test) (FIG. 4). This finding indicates that minocycline does have significant killing effects on persister cells.

Persister cells accumulate more minocycline intracellularly than normal cells.

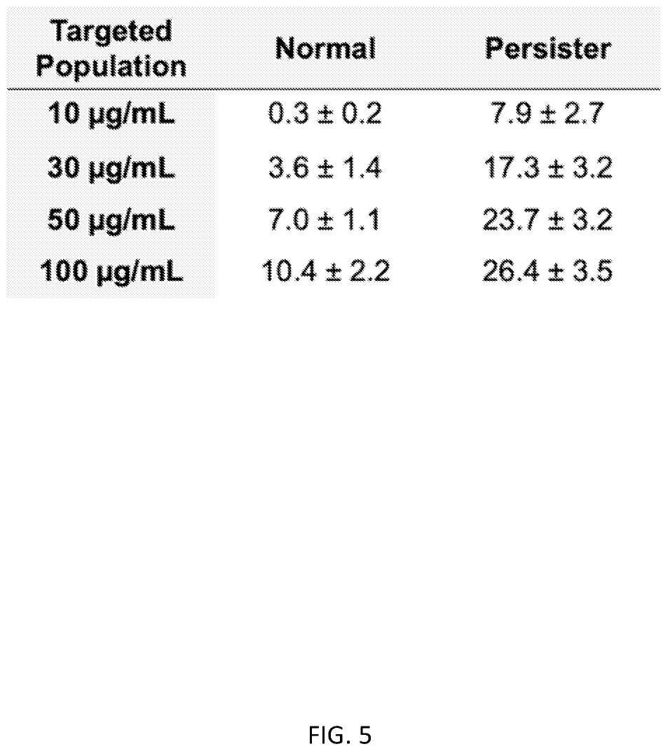

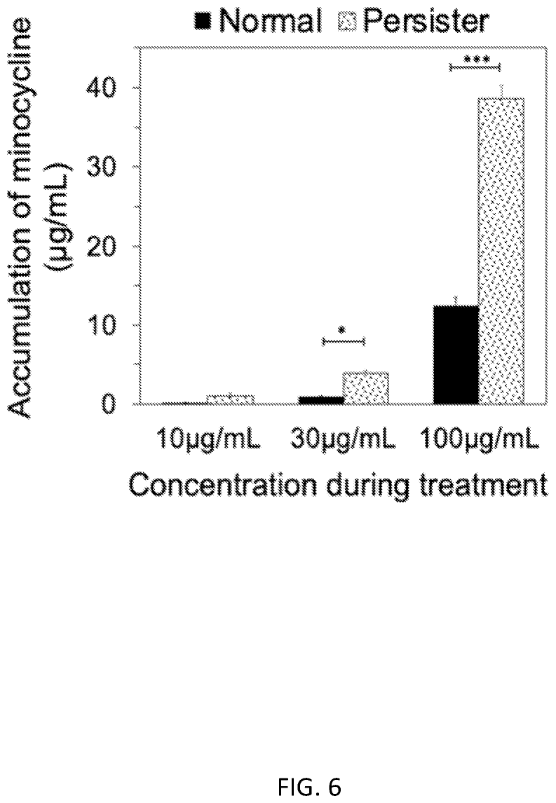

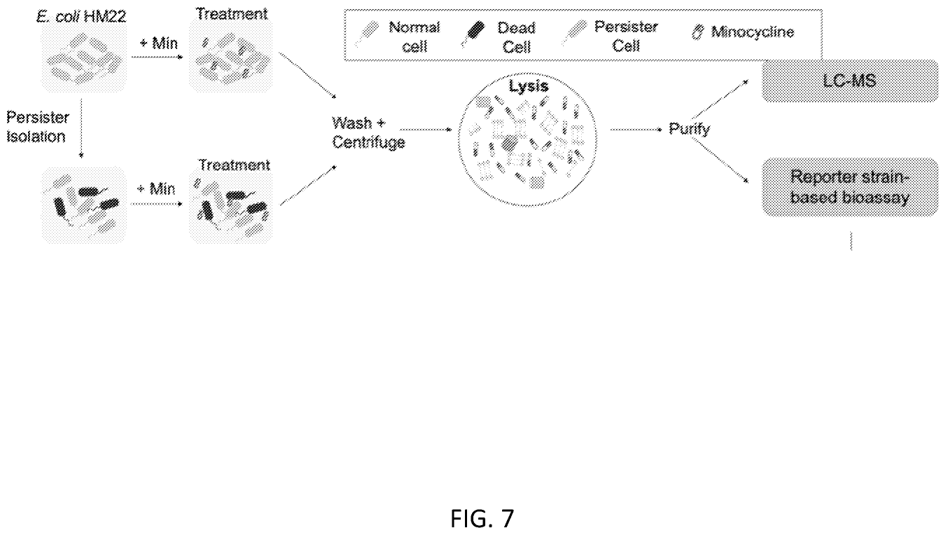

It is interesting that tetracycline and minocycline have different activities against E. coli persister cells although they are from the same tetracycline family. To further understand the stronger killing efficacy of persister cells than normal cells by minocycline, the intracellular concentration of minocycline was quantified in these two populations. Two complementary approaches were used for this test, including a new reporter strain-based bioassay developed recently and conventional LC-MS analysis (FIG. 7 thought 10). The reporter assay is based on Bacillus subtilis 168, which is susceptible to minocycline and allows the quantification of minocycline in E. coli cell lysate by fitting in the standard curve (FIG. 7). Using this assay, the intracellular concentration of minocycline was quantified to be 10.4±2.2 and 26.4±3.5 pg/mL in normal and persister cells, respectively, after treatment with 100 μg/mL minocycline for 1 h (FIG. 5). The findings indicate that E. coli persister cells accumulated ˜2.6 times the intracellular concentration of minocycline compared to normal cells. This finding was corroborated by LC-MS analysis, which revealed persister cells to harbor 3.0±0.4 times the intracellular concentration of minocycline relative to normal cells, after the same treatment (p<0.0001) (FIG. 6). In comparison, the opposite was found for tetracycline. Specially, the intracellular concentration of tetracycline was found to be 5.5±0.1 and 1.6±0.8 pg/mL in normal and persister cells, respectively, after the same treatment at 100 μg/mL.

The difference in antibiotic accumulation between tetracycline and minocycline is not unexpected. Tetracycline uptake can occur by diffusion but mostly through energy-dependent mechanisms while minocycline enters bacterial cells mainly by passive diffusion. In addition, tetracycline has a lower binding affinity to the target compared to minocycline. The dissociation constant of minocycline and 30S ribosome subunit is 3.5×10⁻⁷ M. In comparison, the dissociation constant between tetracycline and its target is 1.3×10⁻⁵ M, approximately two orders of magnitude lower than minocycline. Collectively, the results indicate that persister killing by minocycline but not tetracycline was due to higher accumulation and stronger target binding of minocycline.

Persister cells have reduced efflux activities.

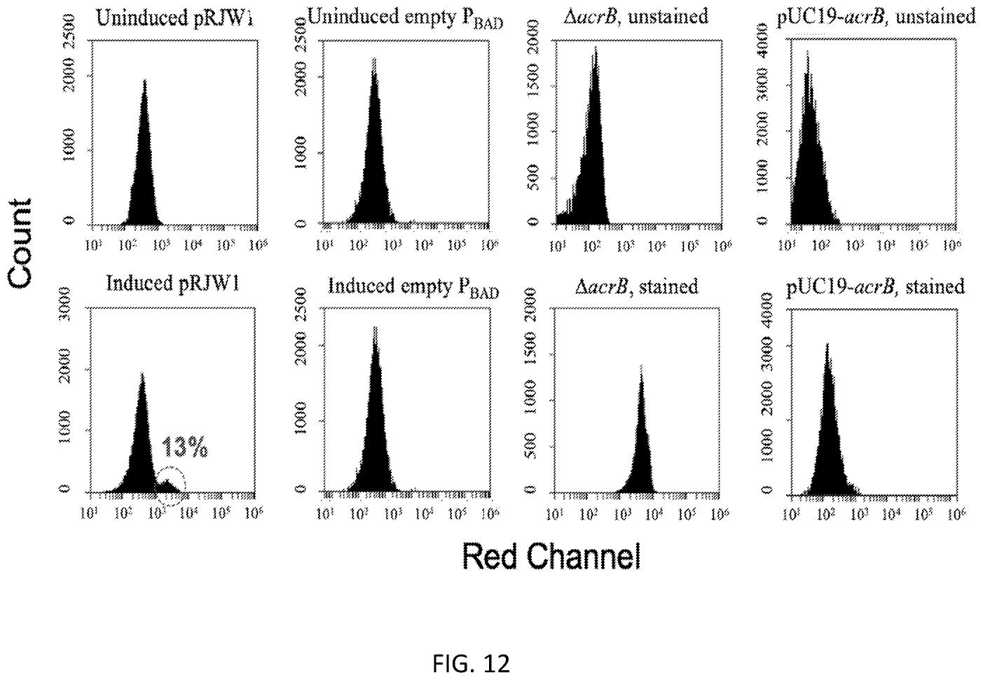

Because minocycline is a substrate of the RND and major facilitator superfamily (MFS) efflux pumps, the present invention originally hypothesized that increased accumulation of antibiotics such as minocycline in persister cells is linked to reduced efflux activity. To test if persister cells have reduced efflux activities, normal and persister cells of E. coli were compared using ethidium bromide (EtBr) staining since the concentration of EtBr in bacterial cells is determined by efflux activities driven by PMF. To avoid the interference of signals from dead cells and cell debris, the P_{BAD} inducible system was used to generate persisters in this experiment rather than persister isolation by killing normal cells using ampicillin. To do so, E. coli Top10/pRJW1 was constructed to allow hipA overexpression under the control of the arabinose-inducible P_{BAD} promotor. The EtBr signal increased in cells exposed to arabinose (to induce hipA expression and thus persister formation) relative to the uninduced samples. Specifically, the EtBr signal was 24.3±3.2%, 29.9±1.6%, and 18.9±1.5% higher in induced relative to uninduced samples after 5, 10, and 30 min of incubation, respectively (FIG. 4a. p<0.001 for all conditions). One-way or Two-way ANOVA followed by Tukey test were used for all analyses unless noted otherwise. The difference in EtBr accumulation decreased after 30 min of incubation possibly due to the toxicity of EtBr. These results were confirmed using flow cytometry (FIG. 12). When the hipA gene was overexpressed, approximately 13% of the population shifted further to stronger red fluorescence compared to the uninduced control (FIG. 12). To verify if this shift was due to decrease in efflux pump activities, the EtBr staining was repeated using an efflux mutant E. coli ΔacrB. As shown in FIG. 3, stained normal cells of E. coli ΔacrB exhibited strong fluorescence (FIG. 12) similar to the brightest subpopulation of induced pRJW1 cells (FIG. 12), which was not observed in the complemented strain (FIG. 12). This finding strongly suggests that persister cells have reduced efflux activities.

### Inactivation of Efflux Pumps Sensitized Normal Cells to Minocycline.

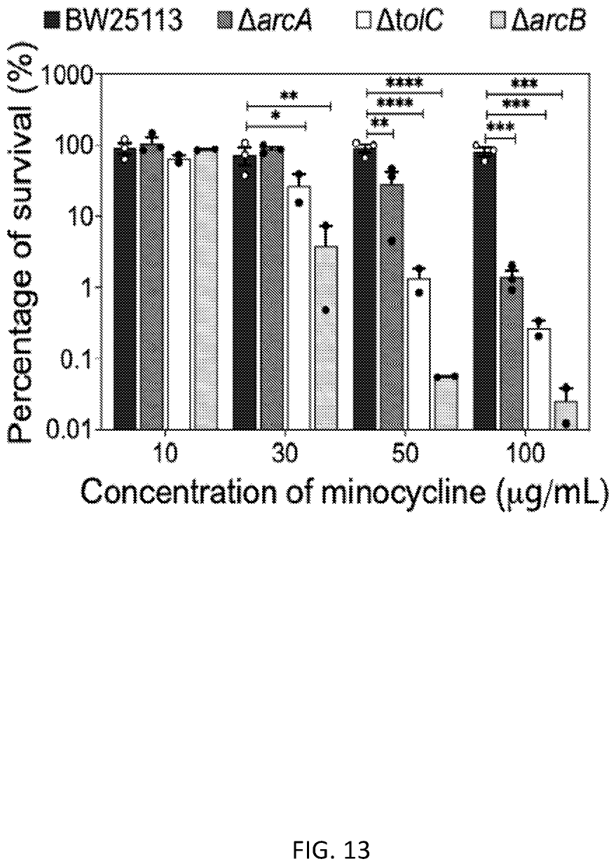

The intrinsic resistance of E. coli normal cells to minocycline involves RND and MFS pumps, which both require PMF to function. Since persister cells that are sensitive to minocycline demonstrated reduced efflux activity, the present invention next asked if inactivating or reducing efflux activity in normal cells renders normal cells as sensitive to killing by minocycline as persister cells. To test this, E. coli JW4364 (ΔacrA mutant), JW5536 (ΔacrB mutant), and JW5503 (ΔtolC mutant) were compared with their wild-type strain E. coli BW25113 for minocycline susceptibility. As expected, increased killing of all three efflux mutants was observed compared to the wild-type strain. For example, 100 μg/mL minocycline killed normal cells of ΔacrA, ΔacrB, and ΔtolC by 98.6±0.3% (p=0.0002), 99.9±0.01% (p=0.0002) and 99.7±0.03% (p=0.0011), respectively; while no significant killing of normal cells of the wild-type strain was observed (FIG. 13). This finding further supports the hypothesis that intercellular antibiotic accumulation increases in persister cells due to the lack of active efflux pumps, leading to enhanced killing.

E. coli persister cells have lower membrane potential than normal cells.

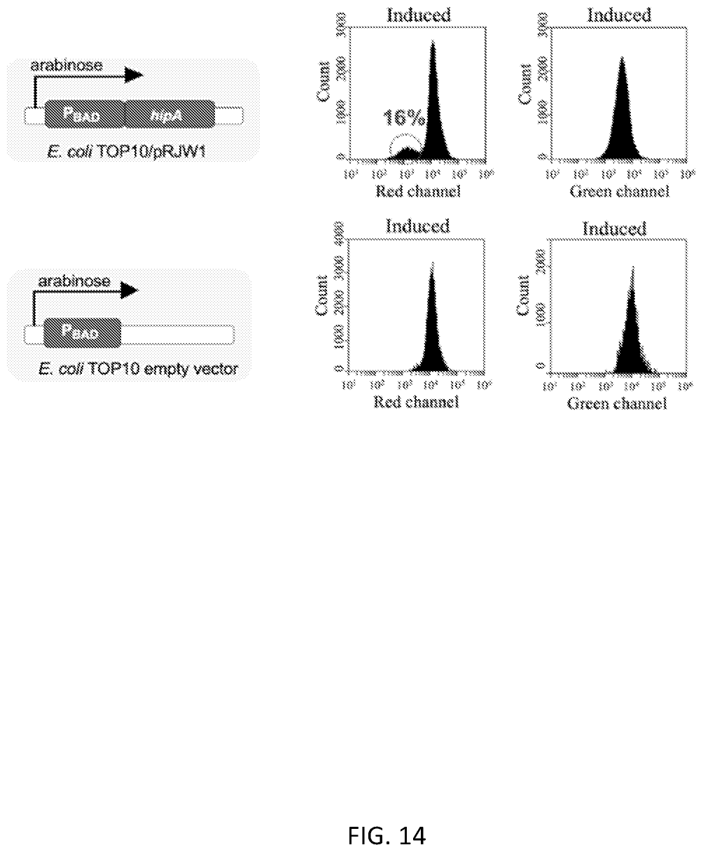

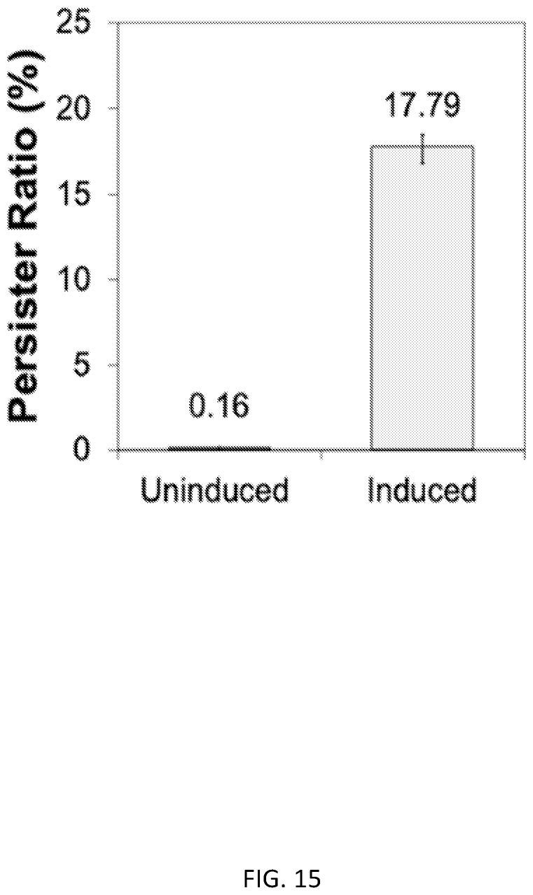

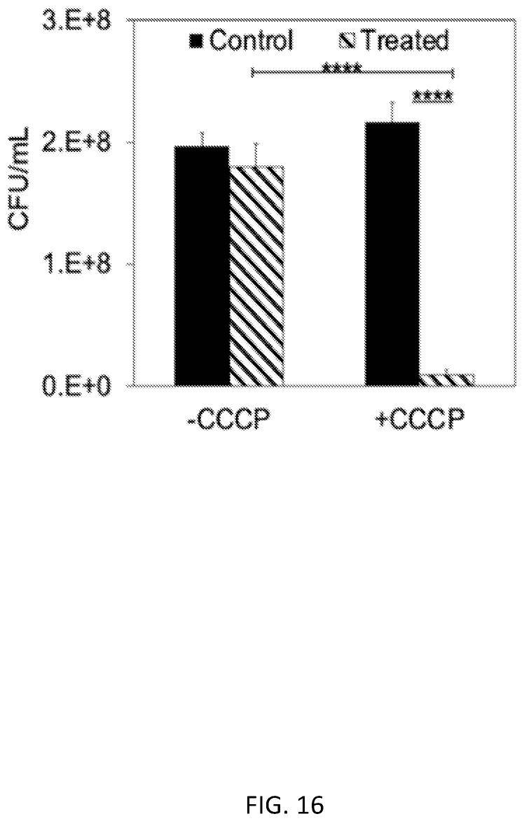

Previous studies have reported the association between persister formation and the reduction in membrane potential. For example, pretreatment with salicylate collapses the membrane potential through the production of ROS; thereby inducing persistence. In addition, increase in Obg levels induces the production of HokB, a small membrane peptide, that induces persistence through pore formation leading to ATP leakage and depolarized membrane. These can explain the reduced efflux activities in persister cells observed in our study. To confirm if our persister cells also have lower membrane potential than normal cells, E. coli Top10/pRJW1 normal and persister cells (induced by overexpressing hipA and pretreatment with 50 μg/mL tetracycline) were compared using JC1, a potentiometric dye that has the ratio of red/green fluorescence positively correlated with membrane potential. Upon induction of persister formation by overexpressing hipA, approximately 16% of the total counts (FIG. 14) exhibited a reduction in the red fluorescence (no change in green fluorescence), while the rest of the population had a strong red fluorescence as observed in the uninduced control (FIG. 15). It is of interest to note that induction of persister formation by hipA overexpression coincided with 17.8±0.6% (FIG. 16) of the induced population as persister cells, as confirmed by CFU counts. This suggests that the shift toward lower red fluorescence, and thus, reduced membrane potential in 16% of the cell population likely occurred in persister cells.

Membrane depotentiation leads to increased killing of E. coli cells by minocycline.

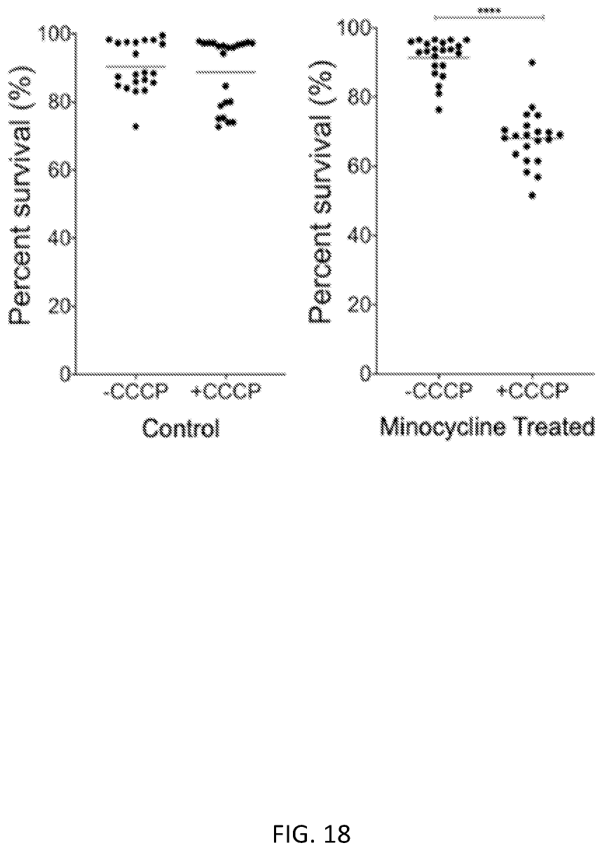

The above results indicated that reduced efflux activity in persister cells can lead to increased accumulation of certain antibiotics like minocycline. If increased accumulation of antibiotics is indeed the cause of perister killing, it was anticipated that membrane depotentiation will likewise sensitize normal cells to minocycline. Because the membrane potential is governed by the PMF and transmembrane pH gradient across the bacterial cell membrane, a reduction in membrane potential indicates reduced PMF which impairs the function of efflux pumps. This leads to increased accumulation of antibiotics that penetrate bacterial membranes without active transport, such as minocycline. Carbonyl cyanide m-chlorophenylhydrazone (CCCP) was used to depotentiate the membrane of E. coli normal cells and thus, mimic the change in membrane potential of persister cells. CCCP dissipates the PMF by allowing protons to leak across the membrane and thus inactivate efflux pumps. Previous studies have shown that CCCP treatment enhances persister formation in E. coli and Pseudomonas aeruginosa. First, the normal cell population was pretreated with 100 μM of CCCP for 10 min, followed by treatment with 100 μg/mL of minocycline for 1 h. Co-treatment of CCCP and minocycline led to 95.9±2.5% (p=0.0146) killing of E. coli normal cells. In contrast, no significant killing by minocycline was observed in the absence of CCCP pretreatment (p=0.9084) (FIG. 16). The CFU results were corroborated by LIVE/DEAD staining that showed significant increase in red fluorescence (propidium iodide stains cells with compromised membranes) among cells treated with minocycline after CCCP pretreatment, while the controls (minocycline alone without CCCP) showed little to no red fluorescence (FIG. 16 through 18).

Killing of persister cells occurs during wake-up.

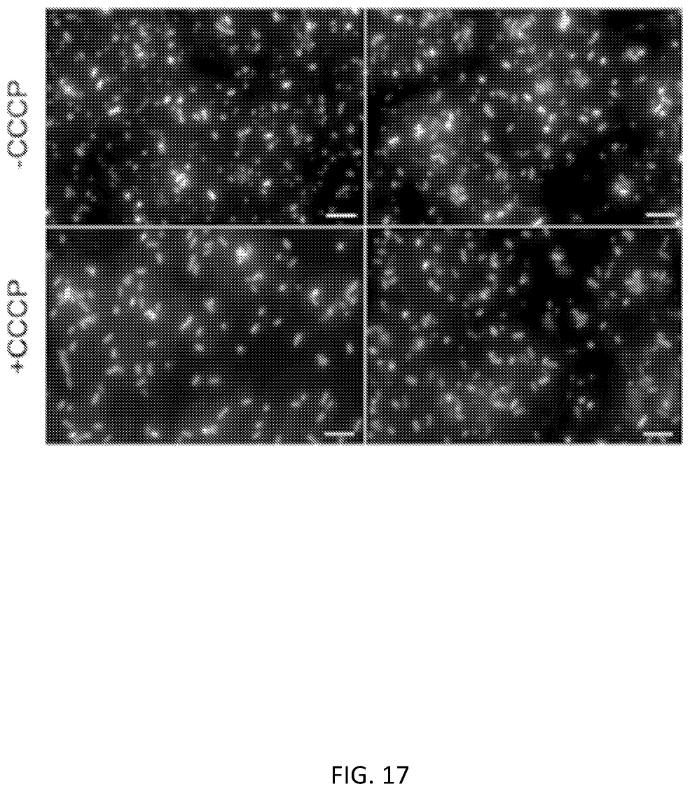

Although persister cells accumulate more minocycline, these cells are dormant and thus lack the growth-associated activities needed to generate corrupted products for killing to occur. The present invention thus hypothesized that the killing effects took place during persister wake-up when the external antibiotic was withdrawn (after the treated cells were plated on antibiotic-free agar plates in this test). Minocycline has a dissociation constant of 3.5×10⁻⁷ M to the 30S subunit of ribosome; thus, it was speculated that when the extracellular concentration of minocycline decreases there is still sufficient intracellular antibiotic concentration to kill these cells upon wake-up. To understand if this occurs, the dynamic change in the viability of E. coli HM22 persister cells was followed after extracellular minocycline was removed, and nutrients were added to “wake up” persister cells. The persister population showed stronger red fluorescence after LIVE/DEAD staining than normal cells in general, presumably due to reduced membrane potential and higher permeability to propidium iodide (FIG. 17). But no significant difference (p=0.4423) in red/total fluorescence ratio was observed before and immediately after minocycline treatment (100 μg/mL in PBS) (FIG. 17 through 19). This result indicates that the killing of persister cells did not occur during the 1 h minocycline treatment.

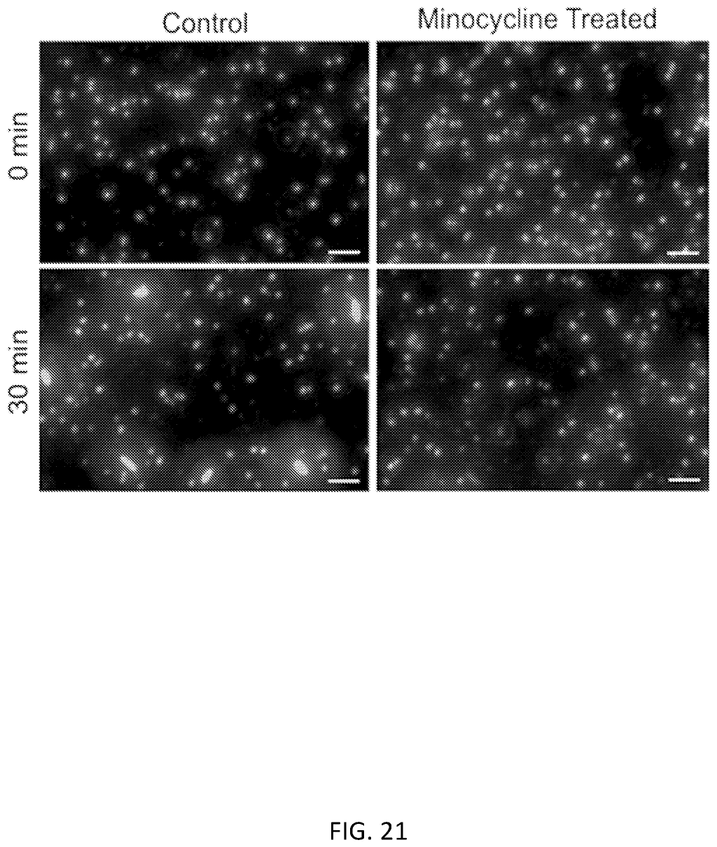

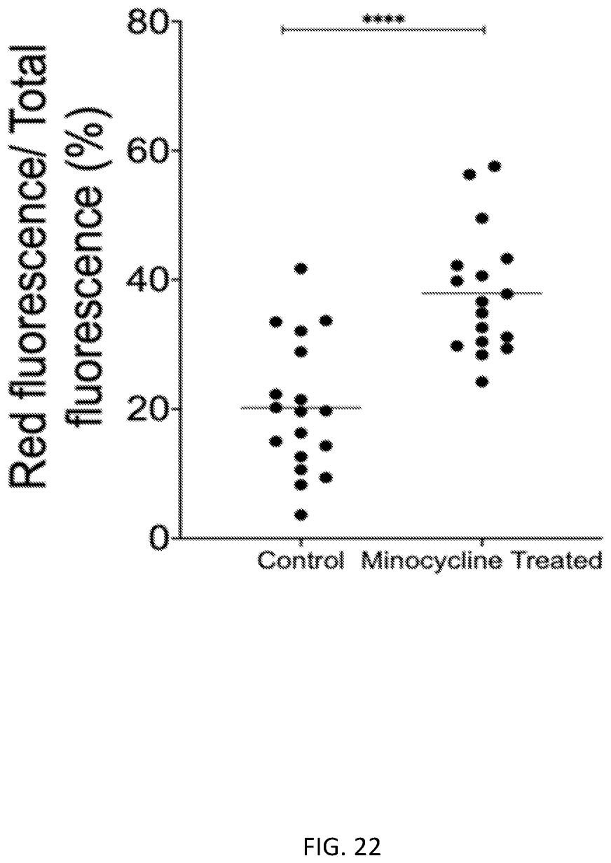

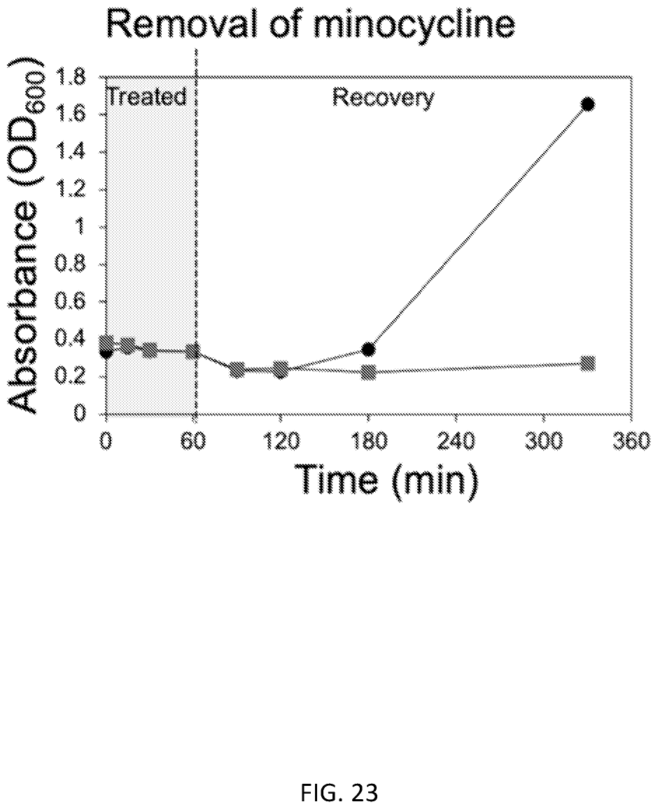

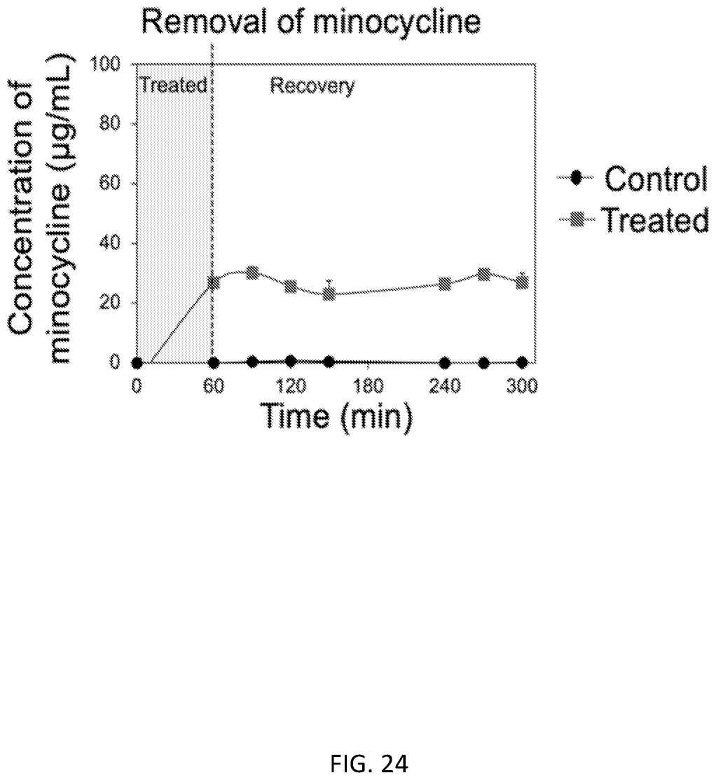

To test if killing occurred during persister wake-up, the untreated and treated samples were then replenished with 500 μL of LB after washing the cells with PBS to remove extracellular minocycline. After 30 min of incubation with added LB, the red/total fluorescence in untreated persisters decreased from 28.3±4.8% to 20.9±3.3%, indicating the cells were waking up. In contrast, the minocycline treated cells exhibited an increase in red fluorescence from 20.2±3.3% to 37.9±3.0%. The different trends between the two groups (FIGS. 21 and 22) indicate that killing occurred during wake-up. Consistently, the untreated persister population gradually regrew with an increased OD₆₀₀ at 2 h after adding LB medium, while the OD₆₀₀ of the treated population remained the same (FIG. 23). This is consistent with the CFU data in FIG. 3 and the fluorescence results in FIG. 22. Using the same reporter assay, it was found that the minocycline concentration remained at 26.8±3.2 pg/mL in persister cells even 4 h after washing the cells and adding LB medium (FIG. 24), essentially unchanged from 26.9±0.7 μg/mL right after the 1 h treatment (FIGS. 24 and 5). Collectively, these results show that removing the extracellular antibiotic and adding nutrients to wake up persister cells can cause killing of this dormant population by internalized antibiotics if there is significant binding to the target.

### New Criteria for Selecting Persister Drugs.

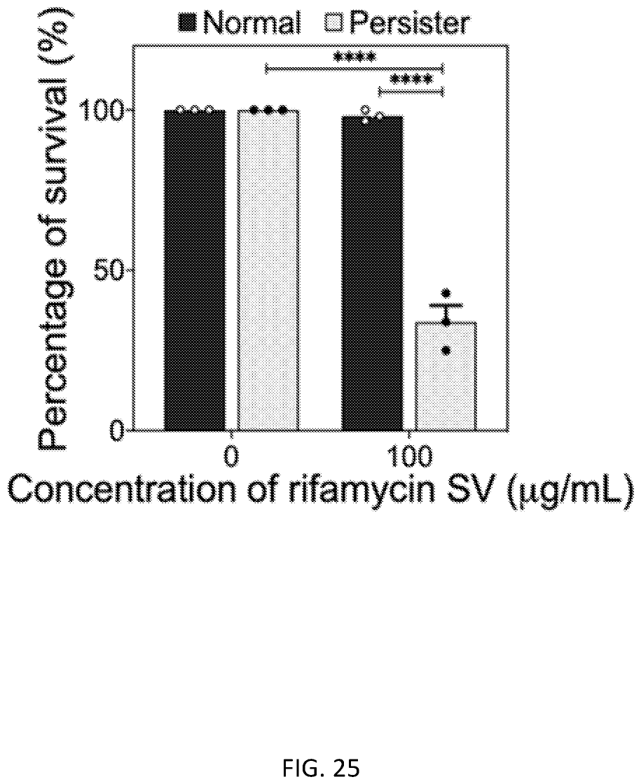

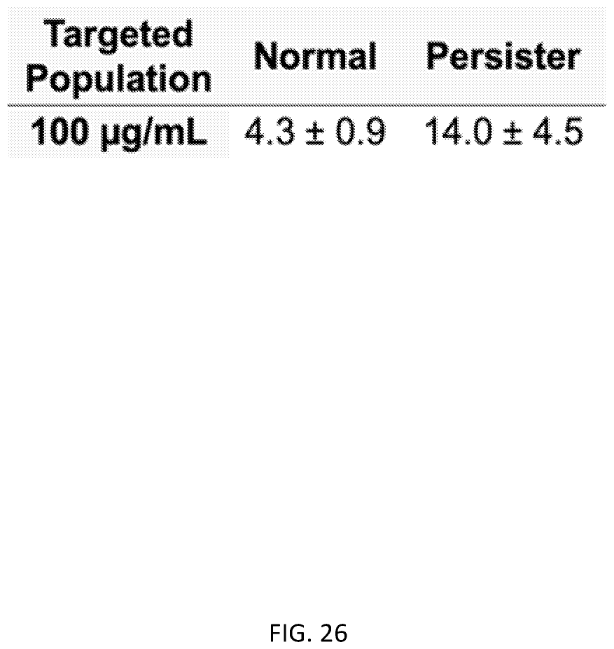

Based on these results, a set of criteria for selecting persister control agents was developed. Specifically, a good persister drug should: (1) be positively charged under physiological condition to interact with the negatively charged lipopolysaccharides on bacterial outer membrane, (2) be amphiphilic to have membrane activity for penetration, (3) be capable of penetration by energy-independent uptake, (4) have strong binding affinity with the target. The first three criteria will ensure effective penetration and accumulation; while (4) is important for killing to occur when persisters wake up with the withdrawal of extracellular antibiotics (killing occurs before the antibiotic is extruded or diffuses out). To validate this strategy, rifamycin SV, a hydrophobic antibiotic that penetrates Gram-negative cells by diffusion through lipid bilayers and targets the RNA polymerase, was tested. In addition, rifamycin SV is a substrate of the RND efflux pump encoded by AcrAB-TolC. The data indicated that 100 μg/mL rifamycin SV did not kill normal cells but significantly killed 75.0±5.12% persister cells (p<0.0001; FIG. 25). In addition, persister cells accumulated 3.2 times the antibiotic compared to normal cells (14.0±4.5 vs. 4.3±0.9 μg/mL) when both populations were treated with 100 μg/mL rifamycin SV (FIG. 26).

Based on these principles, eravacycline, a derivative of minocycline recently approved by FDA in 2018, was tested. Eravacycline also targets the 30S subunit of the ribosome; however, unlike minocycline, the pyrrolidinoacetamido group at C-9 position of eravacycline forms an additional bond with the ribosome. Eravacycline has also been reported to be more potent in inhibiting the ribosomes compared to tetracycline based on their IC50 (concentration of the antibiotic needed to inhibit 50% of the purified 70S ribosome), e.g., 0.2±0.1 μM (eravacycline) vs. 3.0±1.2 μM (tetracycline).

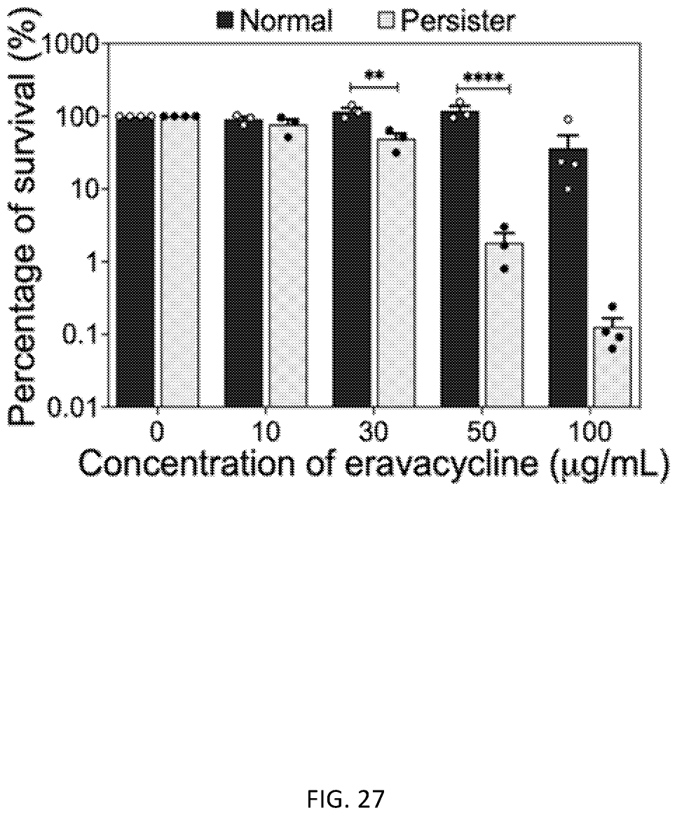

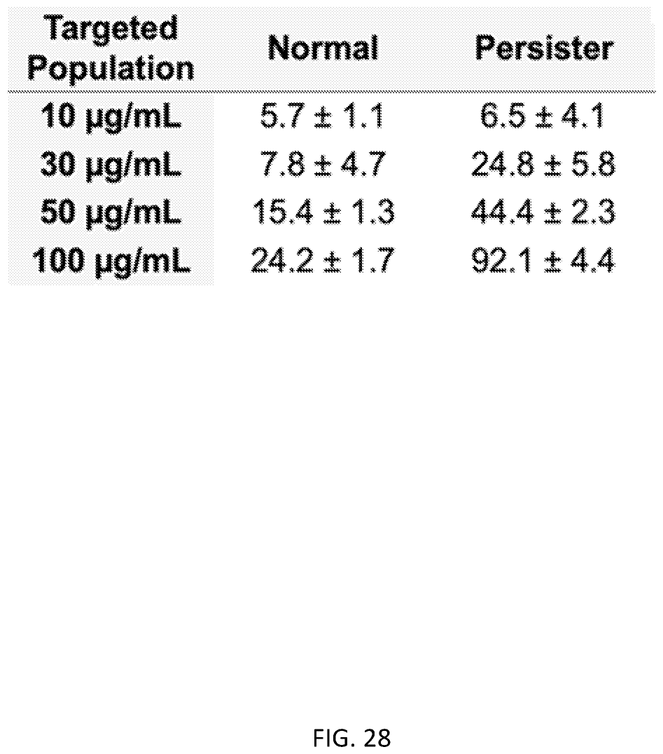

To understand if additional binding to the 30S subunit can increase persister killing, eravacycline was tested at 0, 10, 30, 50, and 100 μg/mL by following the same experimental protocol as other antibiotics in this study. At concentrations between 0-50 μg/mL, there was no significant killing (FIG. 27) of normal E. coli HM22 cells; while it killed 63.5±15.8% at 100 μg/mL (p=0.0043). In a sharp contrast, it killed 50.9±7.7% (p=0.06), 98.2±0.5% (p<0.001), and 99.9±0.03% (p<0.001) of persister cells (FIG. 27) at 30, 50, and 100 μg/mL, respectively. Thus, 3 logs of killing of persister cells was achieved at 100 μg/mL. Similar to minocycline and rifamycin SV, persister cells also accumulated more eravacycline than normal cells. For example, after treatment with 100 μg/mL eravacycline, persister cells accumulated 92.1±4.4 μg/mL, 3.8 times of that in normal cells (24.2±1.7 μg/mL; FIG. 28).

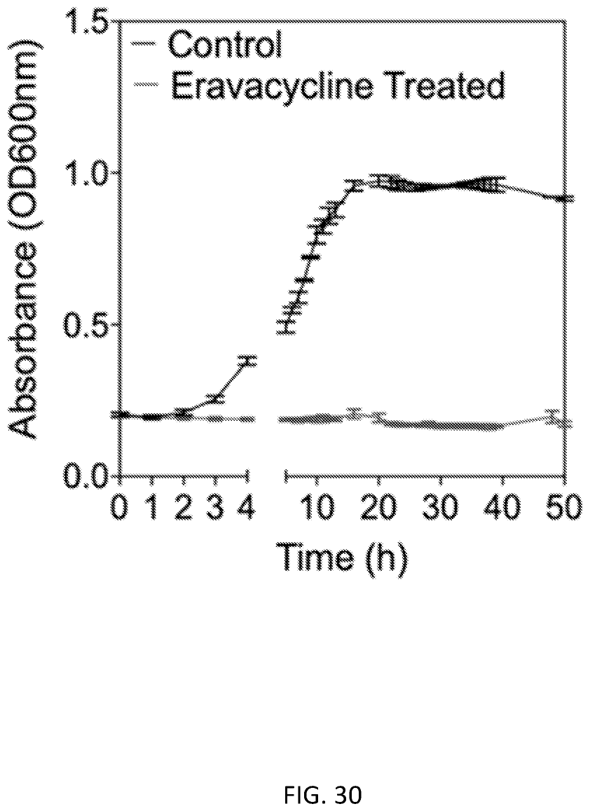

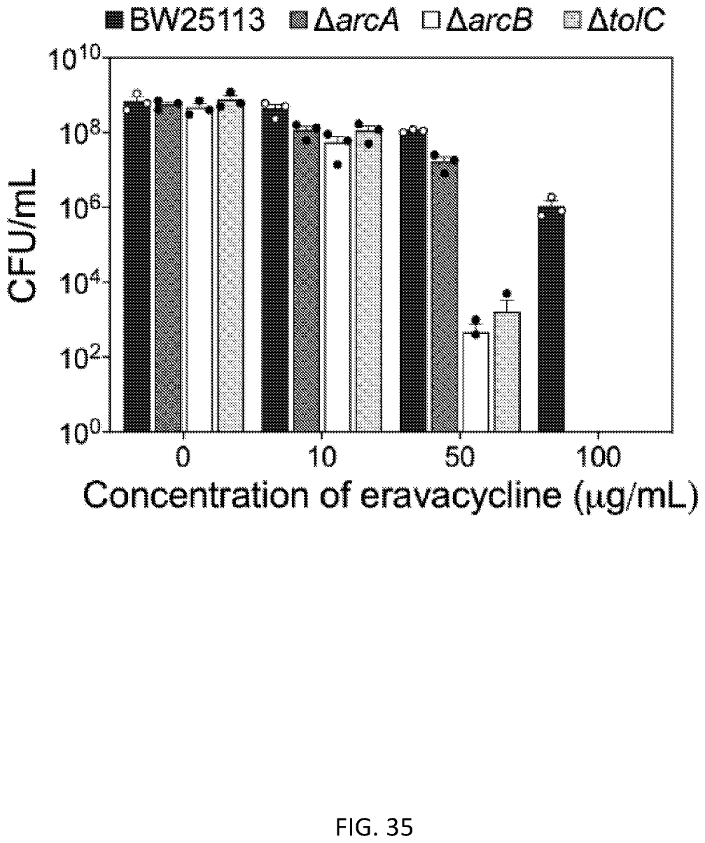

Ampicillin (used to isolate persister cells) was further evaluated to determine if it played a role in the increased killing of persister cells by eravacycline. Similar to the test of minocycline above, concurrent treatment with both 100 μg/mL ampicillin and 100 μg/mL eravacycline caused an additional 99.9% of killing compared to the treatment with 100 μg/mL ampicillin alone (FIG. 30). These results were corroborated by comparing the IC50 (concentration required to kill 50% of the population) of individual treatments vs. co-treatment. The IC50 values were found to be 1.0 μg/mL (ampicillin alone), 4.8 μg/mL (eravacycline alone) and 0.1 μg/mL (concurrent treatment). This leads to a combination index of 0.12, demonstrating strong synergy in bacterial killing. In addition, since both minocycline and rifamycin SV are substrates of the E. coli AcrAB-TolC pumps, eravacycline was tested on AcrAB-TolC mutants and found more killing of normal cells of these mutants compared to the wild-type (FIG. 35).

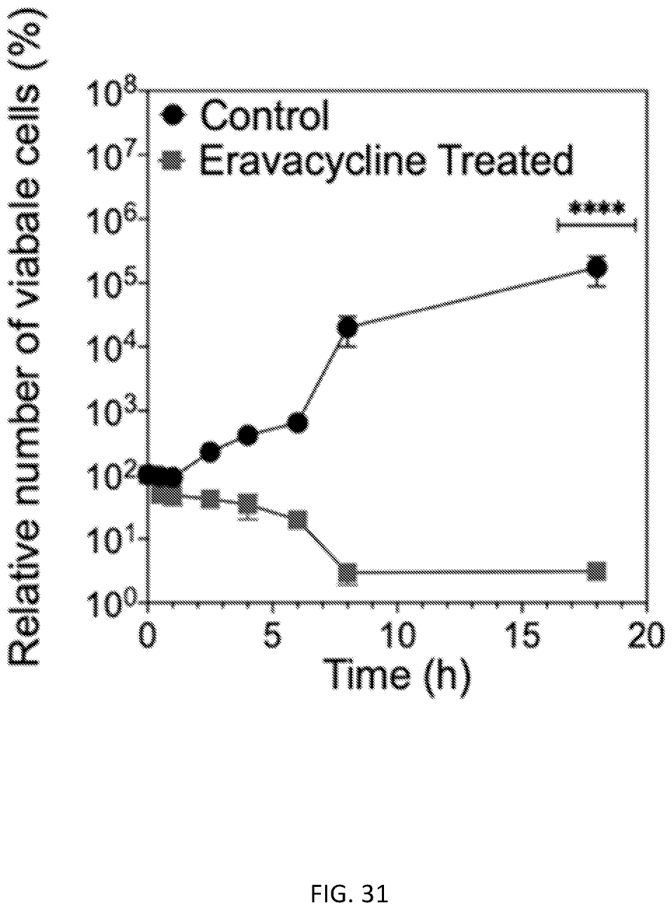

Consistent with the strong target binding of eravacycline, it was found that the treated persister cells were unable to resume growth after removal of extracellular eravacycline and addition of nutrient (CFU continued to decrease by 97.1±0.9% over 8 hours) while untreated persister cells regrew (FIG. 31). Collectively, these results support the proposed criteria and demonstrate that potent killing of persister cells can be achieved by antibiotics that can penetrate bacterial membranes without active uptake and have strong binding to their target.

### Methods

### Bacterial Strains and Growth Media.

Escherichia coli Top10, E. coli HM22 (AT984 dapA zde-264::Tn10 hipA7), E. coli BW25113, E. coli BW25113 ΔacrB, E. coli BW25113 ΔacrA, E. coli BW25113 ΔtolC, S. aureus ALC2085, and Bacillus subtilis 168 were routinely cultured in Lysogeny broth (LB) containing 10 g/L NaCl, 5 g/L yeast extract, and 10 g/L tryptone. E. coli Top10/pRJW1 cultures were supplemented with 100 μg/mL of ampicillin to maintain the plasmid and 0.2% arabinose to induce hipA expression. E. coli HM22 cultures were supplemented with 25 μg/mL diaminopimelic acid (DPA) to ensure its ability to make new cell wall proteoglycan.

### Persister Isolation and Treatment.

Overnight cultures of E. coli HM22 were sub-cultured in LB supplemented with 25 μg/mL DPA with a starting OD₆₀₀ of 0.05 until OD₆₀₀ reached 0.3-0.45. The mid-exponential phase cultures were collected by centrifugation at 13,000 rpm for 3 min at room temperature. The amount of cells used for each treatment was adjusted to OD₆₀₀ of 0.5 in 500 μL. They were then washed with PBS (pH 7.4) three times. For the normal population, the cells were replenished with PBS and immediately treated with minocycline (Sigma Aldrich, St. Louis, MO, USA) for 1 h at 37° C. with shaking at 200 rpm. After 1 h, the treated samples were collected by centrifugation and washed once with PBS to remove the remaining free antibiotic in the solution. The cells were then resuspended in PBS and plated on LB agar plates containing 25 μg/mL DPA to count CFU using the drop plate method. To isolate persister cells, the cells in mid-exponential phase culture were treated with 100 μg/mL ampicillin for 3.5 h at 37° C. with shaking at 200 rpm which resulted in ˜1% of persister cells. After isolation, the cells were washed three times with PBS to remove extracellular antibiotic and then proceeded to minocycline treatment as described above with a starting density of ˜10⁶ cells. Relative viability was normalized by the untreated population. Each experimental condition was tested with five biological repeats. It is important to note that minocycline is both pH sensitive and light sensitive. These factors were considered while performing the assay. The minocycline treatment on E. coli BW25113, E. coli acrB, E. coli acrA, and E. coli tolC mutants were conducted in the same way as described above for the normal population. The tests for eravacycline, tetracycline, chloramphenicol, and rifamycin SV were carried out in the same way as minocycline.

### Quantification of Intracellular Concentrations of Minocycline, Rifamycin SV, and Eravacycline.

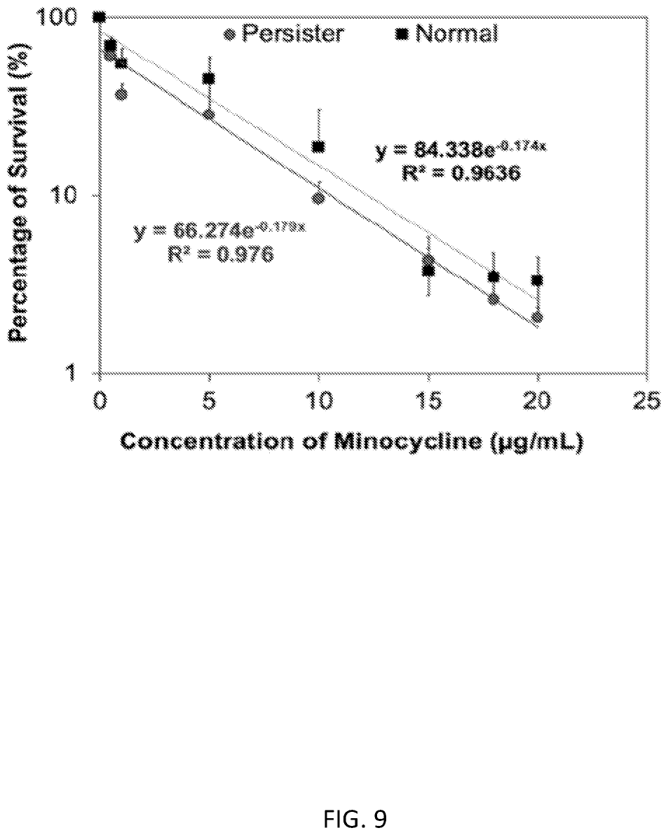

The killing results of the reporter strain treated with E. coli lysate spiked with known concentrations of an antibiotic were used to generate a standard curve (FIGS. 7 and 35) first (E. coli BW25113 ΔtolC for tetracycline, B. subtilis 168 for minocycline and rifamycin SV, and S. aureus for eravacycline), which was then used to determine the concentration in unknown samples. For both populations, the lysates from treated E. coli HM22 cells and untreated controls were collected and dried overnight, and then dissolved in PBS to treat the reporter strain. Cell lysates were extracted using the solvent chloroform after treatment as described above. Then cell debris was removed by centrifugation at 5,000 g for 5 min and the solvent was evaporated overnight in a vacuum desiccator. The samples were concentrated by 5 times before further analysis. For LC-MS analysis: After overnight evaporation as described above, samples were resuspended in 50 μL of DI water. Antibiotics were quantified using a Thermo LTQ Orbitrap mass spectrometer at SUNY Upstate Medical University. A reporter strain-based bioassay was used to corroborate the results. Briefly, lysates extracted with chloroform from both treated and untreated samples were evaporated overnight in a vacuum desiccator after 5× concentration. The evaporated samples were dissolved in 100 μL sterile PBS (pH 7.4) with constant shaking for 5 min using a vortex. The samples were then used to treat the reporter strain with an OD₆₀₀ of 0.5 in 500 μL of PBS. After 1 h of incubation, the treated samples were collected by centrifugation and washed three times with PBS to remove the remaining free antibiotic. The cells were then resuspended with PBS and plated on LB agar plates to count CFU using the drop plate method. Antibiotic concentration was calculated based on the standard curve (FIGS. 3 and 9). Individual cell volume of E. coli HM22 normal and E. coli HM22 persister were calculated based on microscopic images. Total cell numbers were obtained using a hemocytometer for each population.

### Validation of Chloroform Extraction.

To validate if chloroform is effective in extracting the antibiotics after cell lysis, a validation test was performed. Briefly, 100 μL of antibiotic solution was mixed with 100 μL chloroform in a microcentrifuge tube by vortexing. Then the solution was centrifuged for 5 min at 5,000×g. After centrifugation, two distinct phases were seen with the aqueous phase on the top and the chloroform phase at the bottom. Each phase was collected separately and evaporated overnight in a desiccator. On the following day, the evaporated samples were resuspended in 100 μL PBS to dissolve antibiotic with constant shaking for 5 min using a vortex. The samples were then transferred to a 96-well plate where absorbance readings were measured using an Epoch 2 Microplate Spectrophotometer (BioTek, Winooski, VT, USA). Readings for minocycline and eravacycline were taken at 360 nm and 370 nm, respectively. The concentrations were then calculated by comparing with a standard curve of absorbance with known concentrations of corresponding antibiotic. The partition coefficient was calculated based on the concentration extracted from the chloroform phase over the concentration extracted from the aqueous phase (see Table 1 below). Since chloroform was added to the sample as 5:1 (v/v chloroform:aqueous phase), it is estimated that 93.7% of the antibiotic was extracted.

TABLE 1

| | Partition |
|---|---|
| Antibiotic | Coefficient |
| Minocycline | 3.0 ± 0.2 |
| Eravacycline | 3.0 ± 0.3 |

Construction of pRJW1 Carrying P_{BAD}-hipA.

The hipA gene was PCR amplified from E. coli DH5α with added restriction sites of NcoI and EcoRI, included in the forward and reverse primer sequences, respectively. The PCR product was then digested by NcoI and EcoRI and ligated into a similarly digested pBAD/HisD cloning vector to generate pRJW1. The plasmid was then transformed into E. coli Top10 by electroporation.

### Efflux Activity

The results of membrane potential based on JC-1 staining was corroborated by monitoring efflux activities. To induce persister formation, overnight culture of E. coli Top10/pRJW1 was sub-cultured with a starting cell density of 0.01 at OD₆₀₀ (optical density at 600 nm) and incubated till OD₆₀₀ reached 0.15-0.2. This mid-exponential phase culture was supplemented with 0.2% arabinose and incubated for another 3 h at 37° C. with shaking at 200 rpm to induce persister formation through the induction of hipA gene under the P_{BAD} promoter. Induced and uninduced E. coli Top10/pRJW1 cells in exponential cultures were washed and resuspended in PBS as described above. Both samples were stained with 20 μg/mL ethidium bromide (EtBr) and analyzed after 0, 5, 10, and 30 min of incubation to compare the efflux of EtBr. Briefly, excess extracellular EtBr was gently washed away with PBS after staining and 200 μL cell suspension of each sample was transferred to a clear bottomed black walled 96 well plate to measure the signal from EtBr-nucleic acid complex formed in the cells using a microplate spectrophotometer (Model FLx800 microplate reader, Bio-Tek Instruments, Winooski, VT, USA). The JC-1 signal was measured in PBS with excitation at 360 nm and emission at 590 nm.

Meanwhile, a portion of cells from each induced or uninduced population was taken to determine the number of persister cells. These samples were treated with 5 μg/mL ofloxacin for 3 h at 37° C. with shaking at 200 rpm to kill normal cells as described previously. The persister cells harvested by centrifugation were washed with PBS three times to remove any remaining antibiotic in the medium. Then the cells were re-suspended in PBS and plated on LB agar plates to count CFU using the drop plate method as described previously. Each experimental condition was tested with three biological replicates.

### Flow Cytometry.

Flow cytometry analysis was used to corroborate the EtBr efflux results of E. coli Top10/pRJW1. Wild-type E. coli K12 and its efflux pump mutant E. coli ΔacrB were used as positive (low EtBr signal) and negative (max EtBr signal) controls, respectively. The exponential cultures of induced and uninduced E. coli Top10/pRJW1 were stained as described above and the fluorescence signal intensity of each cell in the population was determined using an Accuri™ C6 flow cytometer (BD Biosciences, San Jose, CA, USA).

### Characterizing Membrane Potential.

The membrane potentials of normal (uninduced) and persister (induced) cells of E. coli Top10/pRJW1 were compared using JC-1 potentiometric dye, which is commonly used to stain mitochondria of eukaryotic cells and bacterial membranes based on its membrane potential-induced aggregation (red fluorescence). JC-1 also diffuses into the cytoplasm and emits green fluorescence irrespective of the metabolic stage of a cell; thus, the red/green ratio of JC-1 staining is positively correlated with membrane potential. To induce persister formation, overnight culture of E. coli Top10/pRJW1 was sub-cultured with a starting cell density of 0.01 at OD₆₀₀ (optical density at 600 nm) and incubated till OD₆₀₀ reached 0.15-0.2. This mid-exponential phase culture was supplemented with 0.2% arabinose and incubated for another 3 h at 37° C. with shaking at 200 rpm to induce persister formation through the induction of hipA gene under the P_{BAD} promoter. After 3 h of incubation, 50 μg/mL tetracycline was added and the culture was incubated for another 0.5 h to further induce persister formation by inhibiting protein synthesis as reported previously. Then the cells were collected by centrifuging at 10,000×g for 8 min and washed twice with phosphate buffered saline (PBS). Ten μL JC-1 dye was added in each 300 μL cell sample and mixed by gentle pipetting. The samples were incubated at 37° C. for 15 min in dark. After incubation, excess JC-1 dye was removed by washing with PBS. Then samples were analyzed with flow cytometry to compare membrane potentials by characterizing populations based on red and green fluorescence. Cells emitting high red/green fluorescence ratios were identified as cells with high membrane potential, and vice versa.

### Minocycline Depotentiation Activity.

An overnight culture of E. coli HM22 was sub-cultured in LB medium supplemented with DPA with a starting OD₆₀₀ of 0.05 until OD₆₀₀ reached 0.3-0.45. The mid-exponential culture was collected by centrifugation at a speed of 13,000 rpm for 3 min at room temperature. The amount of cells used for each treatment was adjusted to an OD₆₀₀ of 0.5 in 500 μL LB. The cells were then washed three times with PBS (pH 7.4), and pretreated with 100 μM of CCCP (Sigma Aldrich; dissolved in dimethyl sulfoxide) at 37° C. for 10 min in PBS, followed by immediate treatment with 100 μg/mL of minocycline at 37° C. for 1 h. Then the treated samples were collected by centrifugation and washed once with PBS to remove the remaining free antibiotic. The cells were resuspended in PBS and plated on LB agar plates containing 25 μg/mL DPA to count CFU using the drop plate method. Each experimental condition was tested with three biological replicates.

### Microscopy and Image Analysis.

Treated and untreated samples were washed once with PBS (pH 7.4). Cells were then immediately labeled with LIVE/DEAD BacLight bacterial viability kit (Life Technologies Inc., Carlsbad, CA) with a final concentration of 7.5 μM SYTO9 and 30 μM propidium iodide. After 15 min of staining, the cells were pelleted to remove the staining solution, re-suspended in PBS and vortexed briefly. Labeled cells were then imaged on microscope slides using an Axio Imager M1 fluorescence microscope (Axio Imager M1 fluorescence microscope, Carl Zeiss Inc., Berlin, Germany) with an Orca-Flash 4.0 LT camera (Hamamatsu Photonics, Hamamatsu City, Japan). At least 5 random spots were imaged for each sample. The mean gray value intensity was used to calculate the mean intensity generated from each channel (green and red). Each condition was tested with three biological replicates and 5 images were randomly taken from each sample.

### Antibiotic Diffusion Assay.

Mid-exponential cultures of E. coli HM22 were collected by centrifugation at 13,000 rpm for 3 min at room temperature. The cells were resuspended with LB after washing and then 100 μg/mL ampicillin was added. The samples were incubated for 3.5 h at 37° C. with shaking at 200 rpm to isolate persister cells as described previously. After isolation, the cells were washed three times with PBS to remove the antibiotic and then proceeded to minocycline or eravacycline treatment for 1 h at 37° C. with shaking at 200 rpm. Untreated cells were incubated in the absence of minocycline or eravacycline for 1 h at 37° C. with shaking at 200 rpm. At each designated time point, 1 mL of the cell culture was collected, washed with PBS, and centrifuged at a speed of 13,000 rpm for 3 min. Treated persister cells and untreated controls were washed and resuspended with LB medium supplemented with DPA, and incubated at 37° C. with shaking at 200 rpm. At each designated time point, samples were collected to quantify intracellular antibiotic concentration as described above and to determine growth by measuring OD₆₀₀.

### Statistical Analysis.

Error bars in all figures represent standard error of the mean. All data were analyzed using one-way ANOVA or two-way ANOVA followed by Tukey test if not noted otherwise using SAS version 9.13 (SAS Institute, Cary, NC, USA). Differences with p<0.05 were considered to be statistically significant (*p-value<0.05, **p-value <0.01, ***p-value 0.001 and ****p-value 0.0001).

### Example 2

In another example of the present invention, a chemoinformatic clustering algorithm was optimized based on a new set of criteria to screen persister drugs. This approach was validated using a small chemical compound library (80 compounds) to identify leads that have similar chemical properties of minocycline, which was found effective in killing Escherichia coli persisters. From this screening, it was discovered compound 1b with potent activity against E. coli persister cells, killing 95.5±1.7% of the persister population when treated at 100 μg/mL). This positive hit demonstrates the feasibility of screening for persister drugs using this predictive chemoinformatic model.

By identifying positive candidates (minocycline, rifamycin SV and eravacycline) that can sensitize persister population, it is possible to selectively pick chemical compounds that have similar properties as the positive candidates. Screening chemical library includes thousands of compounds that contains desirable and undesirable compounds. Therefore, there is a need to filter this large library to create subsets with desirable physiochemical parameters that are more similar to the positive candidates. Chemoinformatic clustering algorithm is a powerful tool to analyze large chemical data sets with high dimension. This new high dimensional dataset will be used to group more similar compounds into one cluster. The most popular clustering algorithm is K-means clustering which is an unsupervised learning algorithm that attempts to group samples into a population based on similarity by minimizing the distance between individual data points to the cluster through calculations of centroids (represents the theoretical multidimensional mean of all data points). The screening library from Asinex includes 80 compounds that have been shown to have antibacterial activity with the three additional positive candidates that include minocycline, rifamycin SV and eravacycline.

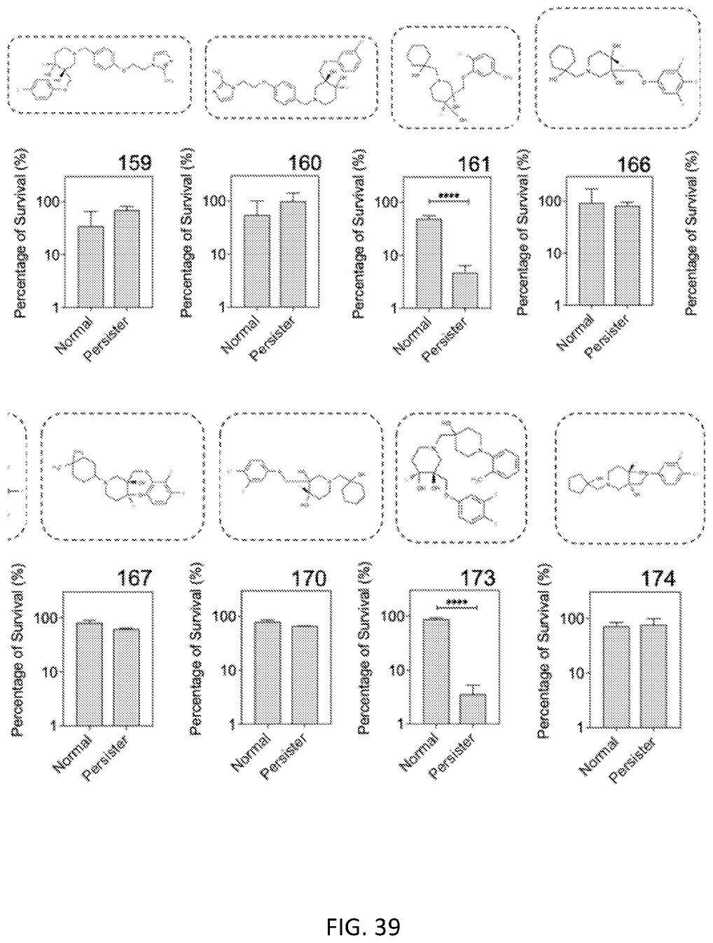

K-means clustering was used to assess the log P (octanol-water partition) and the number of halogen atoms that appear on the compounds. Log P was used as its parameter predicts the interaction of the compound with bacterial outer membrane and correlates with the penetration, as seen in FIG. 39a. Halogens were considered since some known persister control agents contain halogen atom(s), as seen in FIG. 39b. In particular, eravacycline was found to have more potent persister killing activities than its precursor minocycline, which has a fluorine group. The clustering of 80 compounds identified 10 leads that have similar properties to the previous positive candidates, as seen in FIG. 39.

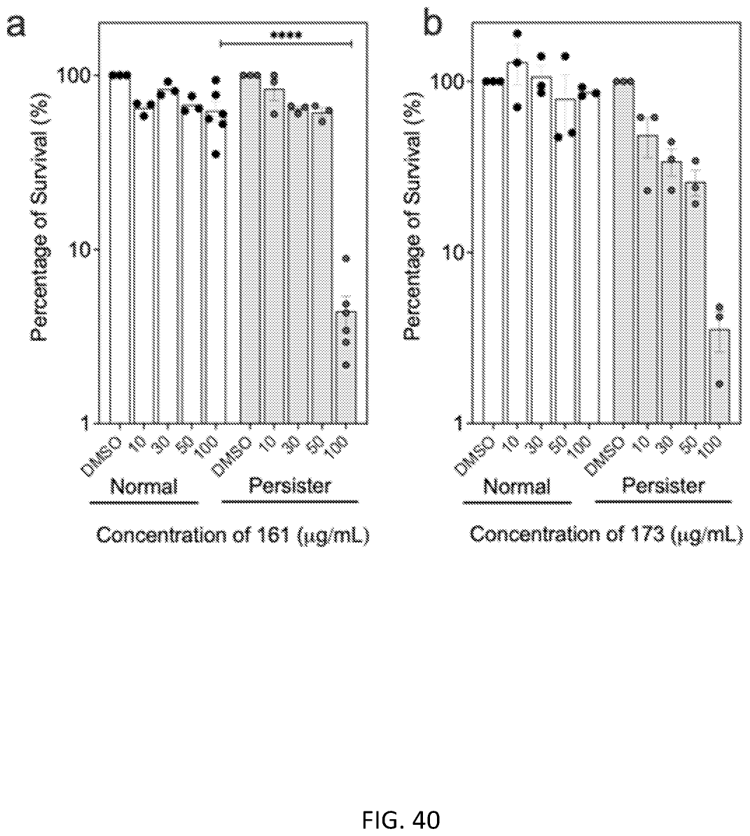

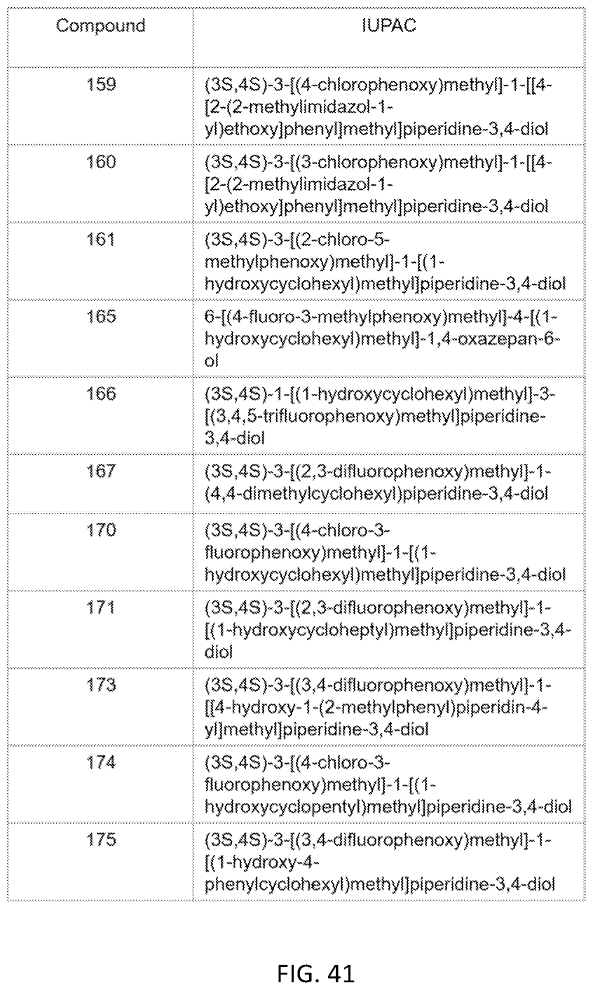

Most of the compounds exhibited no significant killing of either normal or persister cells, as seen in FIG. 40. However, compound 161 showed stronger killing against persister cells then normal cells. It did not show significant killing of normal cells except at the high concentration of 100 μg/mL (by 37.5±0.7%; p=0.003). In contrast, it killed 35.8 0.6% (p=0.02), 38.7±1.3% (p=0.009), and 95.5±1.7% (p<0.001) of persister cells when treated at 30, 50 and 100 μg/mL, respectively, as seen in FIG. 41. For compound 173, no killing of normal cells was observed at concentrations up to 100 μg/mL, but it killed 96.4 0.7% (p<0.01) of persister cells at 100 μg/mL, as seen in FIG. 41.

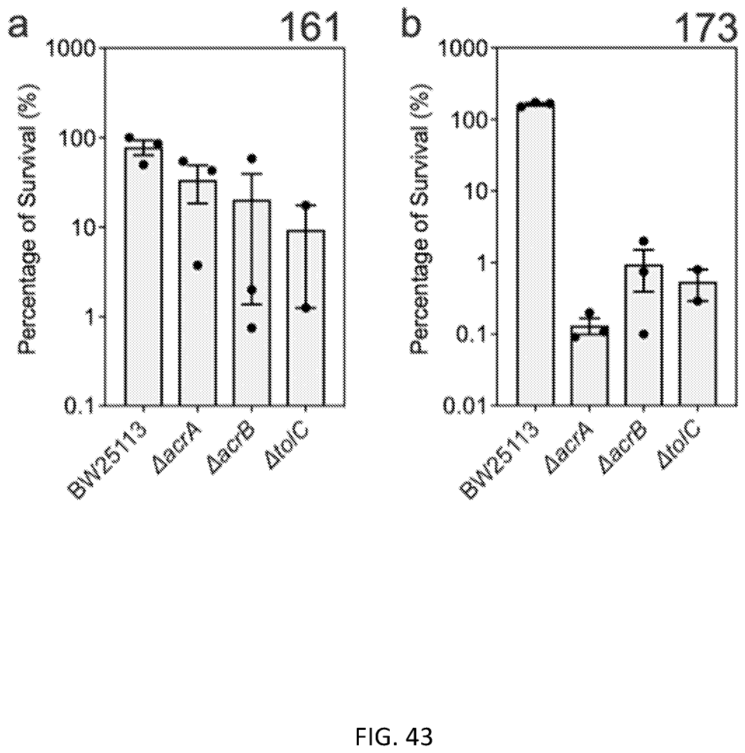

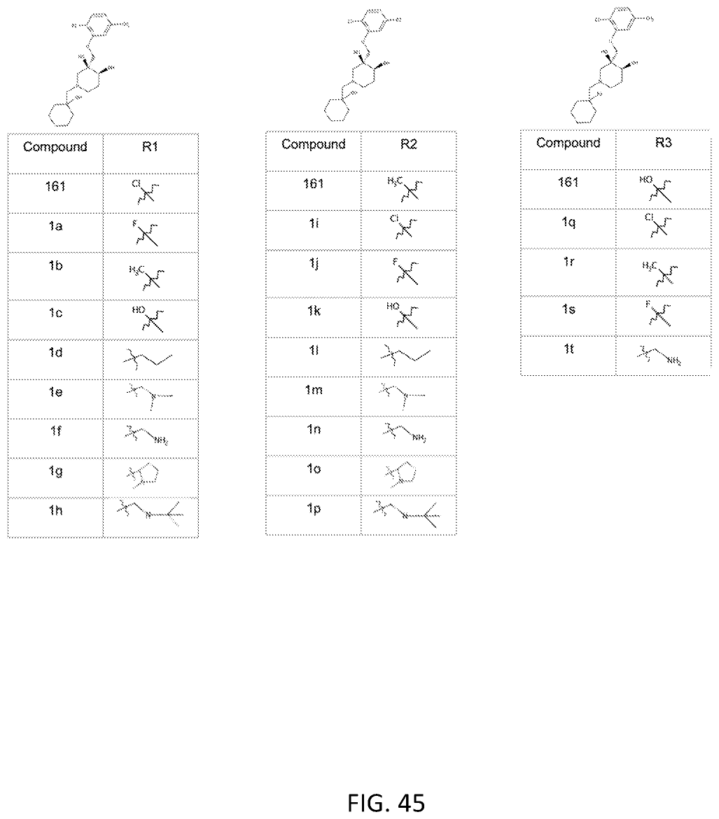

It was then investigated if persister killing was due to reduced efflux of these agents by persister cells. To do this, these compounds were tested to see if they could kill normal cells of multidrug AcrAB-TolC efflux pump mutants, which also have reduced efflux. E. coli JW4364 (ΔacrA mutant), JW5536 (ΔacrB mutant), and JW5503 (ΔtolC mutant) were compared with their wild-type strain E. coli BW25113 for susceptibility. susceptibility. As expected, increased killing of all three efflux mutants compared to the wild-type strain was observed. For example, 50 μg/mL compound 161 killed normal cells of ΔacrA, ΔacrB, and ΔtolC by 66.3±12.0%, 69.8±16.0% and 90.6±4.7%, respectively, while no significant killing of normal cells of the wild-type strain was observed, as seen in FIG. 45. Compound 173 killed normal cells of ΔacrA, ΔacrB, and ΔtolC by 99.9±0.02% (p=0.0001), 99.1±0.5% (p=0.0001) and 99.5±0.1% (p=0.0001), respectively, while no significant killing of normal cells of the wild-type strain was observed. As expected, increased killing of all three efflux mutants by compounds 161 and 173 was observed as compared to the wild-type strain, as seen in FIG. 43. This finding further demonstrates a correlation between the lack of efflux and increase in persister killing.

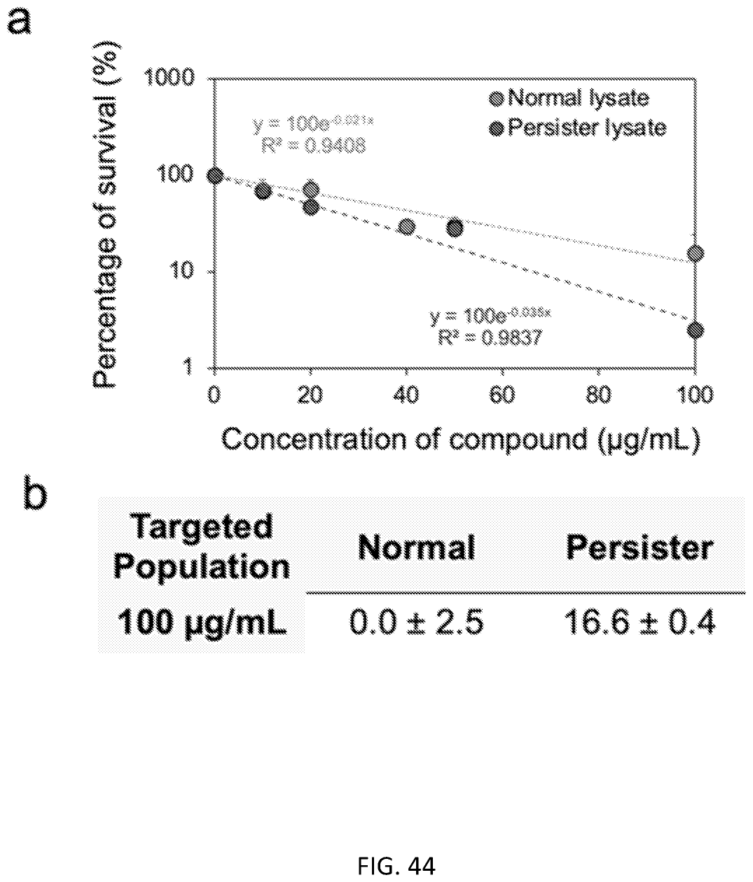

To corroborate the persister killing results, the penetration of compound 161 was investigated as seen in FIG. 44. Consistently, compound 161 showed an intracellular concentration of 1.5±0.4 μg/mL in the normal cells but 16.6±0.4 μg/mL in the persister cells when treated at 100 μg/mL. The higher accumulation in the persister population can help explain the strong killing activities observed. Overall, the results demonstrate that the predictive chemoinformatic model can be used for persister control.

What is claimed is:

1. A method of treating a bacterial infection, comprising the steps of:
administering an antibiotic to a population of bacterial cells including at least some persister cells that are tolerant to antibiotic treatment, wherein the antibiotic is capable of energy-independent diffusion such that the antibiotic does not require active transport to penetrate a membrane of each bacterial cell in the population of bacterial cells; and
waking up the bacterial persister cells in the population of bacterial persister cells after the amphiphilic antibiotic is administered.

2. The method of claim 1, wherein the antibiotic is amphiphilic.

3. The method of claim 1, wherein the antibiotic is selected from the group consisting of minocycline, eravacycline, and rifamycin.

4. The method of claim 1, wherein the step of waking up the bacterial persister cells comprises adding an amount of nutrients to the population of bacterial cells.

5. The method of claim 2, wherein the step of waking up the bacterial persister cells comprises removing any extracellular antibiotics from the population of bacterial cells.

6. The method of claim 1, further comprising the step of administering a second antibiotic, wherein the second antibiotic is able to kill active bacterial cells.

7. The method of claim 6, wherein the step of administering the antibiotic capable of energy-independent diffusion and the second antibiotic requires active transport.

8. The method of claim 6, wherein the two antibiotics are administered at the same time.

9. The method of claim 6, wherein the two antibiotics are administered sequentially with the second antibiotic administered second.

10. A method of screening compounds for efficacy against bacterial persister cells, comprising the steps of:
administering a target compound to a population of persister cells that are resistant to antibiotic treatment;
waking up the bacterial persister cells in the population of bacterial persister cells after the amphiphilic antibiotic is administered; and
observing whether any of the bacterial persister cells are killed after the step of waking up the bacterial persister cells.

11. The method of claim 10, where the step of waking up the bacterial persister cells comprises adding an amount of nutrients to the population of bacterial cells.

12. The method of claim 11, wherein the amount of nutrients comprises a lysogeny broth (LB).

13. The method of claim 12, further comprising the step of selecting the target compound from a library of compounds using physiochemical parameters matching to an antibiotic that is capable of energy-independent diffusion such that the antibiotic does not require active transport to penetrate a membrane of a bacterial cell.

14. The method of claim 13, where the step of selecting the target compound from the library of compounds using physiochemical parameters matching includes using a chemoinformatic clustering algorithm.

15. The method of claim 14, where the chemoinformatic clustering algorithm is K-means clustering.
